(19) United States
(12) Patent Application Publication
(10) Pub. No.: US 2021/0255141 A1
(43) Pub. Date: Aug. 19, 2021

(54) ELECTROSEPARATION SYRINGE AND ANALYTICAL PROCESSES USING THE ELECTROSEPARATION SYRINGE

(71) Applicant: UNIVERSITY OF TASMANIA, Tasmania (AU)

(72) Inventors: Ibraam Emad MIKHAIL, Tasmania (AU); Michael Charles BREADMORE, Tasmania (AU)

(21) Appl. No.: 17/250,215

(22) PCT Filed: Jun. 27, 2019
(86) PCT No.: PCT/AU2019/050675

§ 371 Date: Dec. 16, 2020

(30) Foreign Application Priority Data
Jun. 27, 2018 (AU) 2018902309

(51) Int. Cl. G01N 27/447; C07K 1/24; G01N 27/413; G01N 33/487

(52) U.S. Cl. CPC G01N 27/44795; G01N 33/487; G01N 27/413; C07K 1/24

(57) ABSTRACT

The present application relates to processes for modifying the distribution of a compound in a solution comprising drawing the solution comprising the compound into an electroseparation syringe comprising a syringe barrel, a plunger and electrodes positioned to apply a voltage across solution contained in the syringe barrel, and applying a voltage across the solution in the syringe barrel to modify the distribution of the compound within the solution contained in the syringe barrel. The process may be performed as a part of an analytical process. Also described is an electroseparation syringe for performing such processes, and an apparatus for analysing a sample, the apparatus comprising: —an electroseparation syringe comprising a syringe barrel, a plunger and a pair of electrodes positioned to enable a voltage to be applied across any liquid contained within the syringe barrel, or a receiver for receiving an electroseparation syringe; —a power supply for supplying a voltage potential; —a plunger controller for operation of the plunger to draw up and dispense liquid into the syringe barrel; —an analyser for analysing liquid delivered to the analyser; —a sample reservoir for holding solution to be subjected to analysis; —a valve in fluid connection with the electroseparation syringe that enables fluid flow between the electroseparation syringe and the analyser and fluid flow between the electroseparation syringe and the sample reservoir; and —a controller for controlling operation of the power supply to the electrodes, operation of the plunger to draw liquids into the syringe barrel and dispense liquids from the syringe barrel, and to control the valve setting for controlling the direction of fluid flow.

### TECHNICAL FIELD

This invention relates to processes for modifying the distribution of compounds in solutions, which may form part of analytical processes. The invention also relates to systems involving an electrochemical technique, and equipment used in performing such processes.

### BACKGROUND

Challenges remain in the analysis of analytes, particularly in complex matrices, such as biological samples. For example, in bioanalysis, co-eluting compounds present endogenously in a matrix negatively affect the reproducibility and efficiency of current analytical techniques. This loss of reproducibility and efficiency is commonly referred to as the “matrix effect”. For instance, plasma albumins and different immunoglobulins along with other abundant blood plasma proteins cause a significant ion suppression in electrospray ionization-mass spectrometry (ESI-MS) and consequently interfere with the detection and determination of analytes with less abundance.

Current methods for separating interfering proteins from a plasma sample include protein precipitation (PPT), liquid-liquid extraction (LLE) and solid phase extraction (SPE) techniques. PPT is a nonspecific method based on the low solubility of proteins in aqueous-organic solvent solutions such as aqueous acetonitrile, methanol and acetonitrile-methanol mixtures. LLE involves partitioning an analyte into two immiscible liquids, such as between water or a buffer solution, and an organic solvent such as hexane, diethyl ether, and toluene. SPE relies on the affinity of a particular analyte for a certain stationary phase. According to the characteristics of the analyte and the stationary phase, either the target analyte is retained while the unwanted plasma matrix components are eluted with the solvent, or the interfering matrix components are retained and the target analyte is eluted with the solvent. Optimisation of SPE conditions depends on the physicochemical characteristics of analytes and the nature of matrix components in the sample so typically requires elongated method development. Conventional techniques such as PPT, LLE and SPE are difficult to automate, use large volumes of solvent, are time-consuming and/or frequently involve multiple steps. Accordingly, there is a continuing need for the development of alternative techniques to address the matrix effect.

Electrophoresis is a powerful method of separating molecules based on a suitable property, such as their size, charge or binding affinity to a binding partner (e.g. either a ligand or receptor), under the influence of an electric field or current. Electrophoresis has been employed in the analysis of various analytes, most notably large biomolecules such as peptides or proteins. Electrophoretic techniques include capillary electrophoresis (CE), micellar electrokinetic chromatography (MEKC), gel electrophoresis and microchip electrophoresis. However, electrophoresis also suffers from the matrix effect for some complex matrices.

Isoelectric focusing (IEF) is a technique for separating amphiphilic molecules according to their isoelectric points (pl) under the influence of the applied voltage. The most common type of IEF is carried out by incorporating carrier ampholytes (CAs) in gels or solutions to create a pH gradient. However, CAs are expensive and are incompatible with mass spectrometry (MS) analysis without either a special interface or CA removal before analysis.

Methods of generating pH gradients without the use a CA have been explored. These methods are sometimes referred to as CA-free isoelectic focussing (CAF-IEF) strategies. CAF-IEF techniques have been applied to a range of coupled mass spectrometry (MS) processes and lab-on-a-chip applications.

One CAF-IEF strategy involves the concentration and separation of amphiphilic molecules from their matrix using controlled flows of H⁺ and OH⁻ ions from electrode chambers at opposite sides of a separation column. The solvolytic ions flow toward the centre of the separation column and react to reform water molecules. The flow of H⁺ ions creates a zone of low pH, and the flow of OH⁻ ions creates a zone of high pH. The zone where the reaction of H⁺ and OH⁻ ions occurs experiences a sharp change of pH, and typically has a roughly neutral pH (˜pH 7). This neutral zone is sometimes referred to as a neutralization reaction boundary (NRB). Amphiphilic molecules with pl values between the pHs of the zones of high/low pH surrounding the NRB, are focused into the NRB. Ampholytes focussed in the NRB could then be further separated using the separation column alone, or in a further separation step, such as capillary electrophoresis, or incorporated into a matrix suitable for matrix-assisted laser desorption ionisation time-of-flight (MALDI-TOF) mass spectrometry.

Lab-in-a-Syringe (LIS) systems are a recent approach for integrating different analytical steps within a syringe. Recently, a LIS system was introduced that integrated an automated dispersive liquid-liquid microextraction LIS system with a built-in spectrophotometric detection for determination of rhodamine B in water samples and soft drinks. LIS systems are already considered valuable tools for the target analytes pre-concentration prior to coupling with various analytical techniques such as electrothermal atomic absorption spectrometry, inductively coupled plasma spectrometry, and gas chromatography-mass spectrometry. Also, gold nanoparticle-based LIS systems were developed for immunosensing biomarkers and colorimetric discrimination of alanine enantiomers. Current LIS systems are mainly based on liquid-liquid extraction (LLE) techniques, which suffer from a number of drawbacks, including excessive use of organic solvents, labour intensity, requirement of emulsion formation, and difficulty of automation. None of the current LIS systems are based on electroseparation techniques.

It would therefore be advantageous to provide a LIS system that employs an electroseparation technique. It would also be advantageous to provide an alternative CAF-IEF strategy within a LIS system. It would be further advantageous to provide a process utilising an electroseparation technique that may provide a useful alternative for analysis of biological samples. It would also be advantageous for embodiments of the invention to provide a LIS system capable of separating net neutral molecules from charged species prior to analysis.

### SUMMARY

The inventors have developed analytical processes and systems utilizing an electroseparation syringe. The processes and systems can be used to more effectively analyse amounts of analytes in a solution. These processes and systems may enable accurate and reproducible analyses of lower concentrations of analytes than existing systems and methodologies, or the analysis of smaller amounts of a sample. The processes and systems utilize an electrochemical technique adapted to be carried out within the electroseparation syringe. The technique may involve a separation step where charged molecules are separated from net neutral molecules before injection into an analyser. This separation may be particularly advantageous in the analysis of analytes in complex matrices, such as in the analysis of biological samples. For example, the separation step can be employed to separate interfering charged species from a net neutral analyte, reducing the impact of the matrix effect and allow for more reproducible analyses of the analyte with enhanced sensitivity. The processes and methods may also be used to focus the concentration of an analyte (either charged or net neutral) into a region of a solution, assisting to increase the sensitivity of detection by an analyser.

According to one embodiment, there is provided a process for modifying the distribution of a compound in a solution comprising:
drawing the solution comprising the compound into an electroseparation syringe comprising a syringe barrel, a plunger and electrodes positioned to apply a voltage across solution contained in the syringe barrel, and
applying a voltage across the solution in the syringe barrel to modify the distribution of the compound within the solution contained in the syringe barrel.

The compound may be an analyte. The compound may be described as an analyte if it is subjected to analysis in a subsequent stage of the process.

Modifying the distribution of the compound, or analyte, refers to changing the distribution away from a uniform distribution or dispersion of the compound across the whole of the volume or aliquot of solution in the syringe barrel, or modifying the distribution of other components in the solution so as to change the relative amount (i.e. distribution) of the compound (analyte) with reference to other substances present in the solution. This may be by way of increasing the concentration one region (i.e. zone, area or portion) of the solution and decreasing the concentration in another region of the solution. To provide a number of non-limiting examples, the modifying of the distribution of the compound (e.g. analyte) within the solution contained in the syringe barrel may comprise:
focusing a concentration of the compound (e.g. analyte) within a region of the solution contained in the syringe barrel, or
generating a region within the solution with an increased concentration of the compound (e.g. analyte) in the solution, wherein the compound (analyte) is a net neutral molecule; or
separating the compound (e.g. analyte) from net neutral compounds also contained within the solution, wherein the compound (analyte) is a charged compound; or
where the solution comprises a compound (e.g. “matrix compound(s)”) and an analyte, focusing a concentration of the compound within one region of the solution to create another region of solution containing analyte with a depleted amount of the compound.

Each of the above four examples is the subject of a separate embodiment of the invention.

As the last example demonstrates, the compound that is focused or concentrated in one region of solution need not necessarily be the analyte, and the analyte that is to be analysed may be another species in the solution that remains uniformly distributed in the solution after the application of the voltage across the solution. In this example, another compound or compounds present in the solution, which may be described as matrix compound(s), are re-distributed, so that there is a region comprising analyte in the original concentration in one region with a higher concentration of the matrix compound(s), and another region comprising the analyte in the original concentration with a lower concentration of the matrix compound(s). This form of “cleaning up” the sample to improve ability to analyse the analyte through reducing the concentration of other compounds can also assist with analysis of small amounts of an analyte in a complex (multi-component) sample.

The process may further comprise:
injecting the solution into an analyser to allow analysis of the analyte (e.g. for determining a concentration of the analyte).

The solution in which the compound or analyte is present when the process is performed may be described as a conducting solution. The solution in typical embodiments is an aqueous solution. The solution may comprise a combination of an aqueous solvent (i.e. water) and one or more organic solvents (e.g. acetonitrile). It is possible in alternative embodiments for the solution to be an organic solution, comprising a polar organic solvent.

In one aspect, the invention provides a process for determining a concentration of an analyte in a solution, comprising:
a. applying a voltage across the solution comprising the analyte and a background electrolyte in an electroseparation syringe, the electroseparation syringe comprising a syringe barrel, a plunger and a pair of electrodes positioned to apply the voltage across the solution in the syringe barrel; and
b. injecting the solution into an analyser.

In a specific example of this aspect, the present application provides a process for determining a concentration of an analyte in an aqueous solution, comprising:
a. applying a voltage across the aqueous solution comprising the analyte and a background electrolyte in an electroseparation syringe, the electroseparation syringe comprising an anode and a cathode positioned to apply the voltage across the aqueous solution; and
b. injecting the aqueous solution into an analyser.

In another aspect, the invention provides a process for focussing a concentration of a molecule in a solution, comprising: applying a voltage across the solution comprising the molecule and a background electrolyte in an electroseparation syringe to generate a region comprising an increased concentration of the molecule in the solution, the electroseparation syringe comprising a syringe barrel, a plunger and a pair of electrodes positioned to apply the voltage across the aqueous solution.

In a specific example of this aspect, there is provided a process for focussing a concentration of a net neutral molecule in an aqueous solution, comprising applying a voltage across the aqueous solution comprising the net neutral molecule, one or more charged molecules and a background electrolyte in an electroseparation syringe to generate a region comprising an increased concentration of the net neutral molecule in the solution, the electroseparation syringe comprising an anode and a cathode positioned to apply the voltage across the aqueous solution.

In a further aspect, the present application provides a process for separating a charged compound from a net neutral compound (e.g. an amphiphilic compound), the process comprising applying a voltage across a solution comprising the charged compound, the net neutral compound (or amphiphilic compound) and a background electrolyte in an electroseparation syringe comprising a syringe barrel, a plunger and a pair of electrodes positioned to apply the voltage across the aqueous solution.

In a specific example of this further aspect, the present application provides a process for separating a charged compound from a net neutral compound, the process comprising applying a voltage across an aqueous solution comprising the charged compound, the net neutral compound and a background electrolyte in an electroseparation syringe comprising an anode and a cathode positioned to apply the voltage across the aqueous solution.

In some embodiments of these processes, the solution is an aqueous solution. In some embodiments, the aqueous solution is a biological solution.

In another aspect, the invention provides an electroseparation syringe, comprising a syringe barrel, a plunger, and a pair of electrodes, wherein the electrodes are configured to come into electrical contact with a solution contained within the syringe barrel in use, so as to enable a voltage to be applied longitudinally across solution contained in the syringe barrel.

Expressed another way, the electroseparation syringe of the present invention may comprise a barrel having a discharge end and a receiving end; a plunger; a cathode comprising a first power supply connector; and an anode comprising a second power supply connector, wherein the cathode and the anode are configured to provide a voltage across a solution contained in the barrel.

One electrode may be in the form of a conductive metal needle that is connected to one end of the syringe barrel. The other electrode may be in the form of a conductive metal plunger that is at the opposite end of the syringe barrel to the needle, the conductive metal plunger configured so as to provide direct contact to the interior of the syringe barrel, so that in use, solution contained within the syringe barrel is in direct contact with the conductive metal plunger. As a consequence, there is an absence of electrically conductive seal material (in at least one region) that would normally electrically isolate the plunger from solution held within the syringe barrel.

The electroseparation syringe may be supplied in parts or in whole. A subset of the parts required to make up the electroseparation syringe may be supplied. Thus, in one example, there may be provided an electroseparation syringe kit comprising a syringe barrel comprising a needle connector for connection to a needle, and a plunger that comprises an electrode, the plunger being configured so that when it is positioned in use in the syringe barrel, there is direct electrical contact between solution contained within the syringe barrel and the electrode of the plunger. The kit may further comprise the needle, or the needle may be supplied separately. The needle may comprise a second electrode.

In still a further aspect, the invention provides an analytical system, comprising:
an electroseparation syringe comprising a syringe barrel, a plunger, and a pair of electrodes positioned to apply a voltage across an aqueous solution contained in the syringe barrel in use;
a power supply configured to connect with the electrodes; and
an analyser adapted to receive and analyse an analyte from the electroseparation syringe.

Using alternative language, this system comprises:
an electroseparation syringe comprising an anode and a cathode positioned to apply the voltage across an aqueous solution contained in the electroseparation syringe;
a power supply configured to connect with the anode and the cathode; and
an analyser adapted to receive and analyse an analyte from the electroseparation syringe.

In another aspect, the invention provides a system comprising:
a receiver for an aqueous solution injected from an electroseparation syringe comprising an anode and a cathode positioned to apply the voltage across the aqueous solution when contained in the electroseparation syringe; and
a power supply configured to connect with the anode and the cathode.

In another aspect, the invention provides an apparatus for analysing a sample comprising:
an electroseparation syringe comprising a syringe barrel, a plunger and a pair of electrodes positioned to enable a voltage to be applied across any liquid contained within the syringe barrel, or a receiver for receiving an electroseparation syringe;
a power supply for supplying a voltage potential;
a plunger controller for operation of the plunger to draw up liquid into the syringe barrel and to eject liquid from the syringe barrel;
an analyser for analysing liquid delivered to the analyser;
a sample reservoir for holding solution to be subjected to analysis;
a valve in fluid connection with the electroseparation syringe, that enables fluid flow between the electroseparation syringe and the analyser, and fluid flow between the electroseparation syringe and the sample reservoir; and
a controller for controlling operation of the power supply to the electrodes, operation of the plunger to draw liquids into the syringe barrel and eject liquids from the syringe barrel, and to control the valve setting for controlling the direction of fluid flow.

In a further aspect, the invention provides a reagent kit comprising:
(i) a predetermined amount of a background electrolyte selected from an ammonium salt, a carboxylic acid, a carboxylate salt and an amine or a combination thereof, and
(ii) a predetermined amount of a reference analyte;
and optionally
(iii) a predetermined volume of solvent.

Before describing the present invention in detail, it is to be understood that this invention is not limited to particularly exemplified embodiments, which may, of course, vary. The invention(s) described and claimed herein have many attributes and embodiments including, but not limited to, those set forth or described or referenced in this summary section, which is not intended to be all-inclusive. The invention(s) described and claimed herein are not limited to or by the features or embodiments identified in this summary section, which is included for purposes of overview illustration only and not limitation.

It is to be understood that, if any prior art publication is referred to herein, such reference does not constitute an admission that the publication forms a part of the common general knowledge in the art, in Australia or any other country.

### BRIEF DESCRIPTION OF THE DRAWINGS

The invention will be further described, by way of example only, with reference to the accompanying drawings, in which:

FIG. 1 shows a schematic of the mechanism of formation of a NRB within an electroseparation syringe.

FIGS. 2a and 2b show (a) a schematic of a system comprising an electroseparation syringe of one embodiment of the invention coupled with an electrospray ionisation-mass spectrometer (ESI-MS), and (b) a more detailed schematic illustration of the electroseparation syringe.

FIG. 3 shows a series of images showing the change in colour pattern of a universal indicator in an electroseparation syringe over time under electrolytic conditions.

FIGS. 4a and b show (a) a series of images of the isoelectric focusing (IEF) of BSA (pl 4.7, 100.0 μg/mL) labelled with 2 different Chromeo™ dyes within an NRB within five minutes of voltage application and (b) a series of images showing the ability of the developed processes to focus different proteins; R-phycoerythrin (RPE-pl 4.2), (40.0 μg/mL) and haemoglobin (HGB-pl 6.9), (350.0 μg/mL), labelled with a mixture of Chromeo™ 488 labelled BSA (100 μg/mL) and HGB (350.0 μg/mL).

FIGS. 5a and b show (a) a plot of peak heights (EIEs; m/z 156.0±0.1) of unspiked and spiked urine samples with final added concentrations of 4.0, 8.0, and 16.0 μg/mL histidine obtained by the process of Example 3; and (b) a quadratic fit calibration curve with the integrated equation for the estimation of histidine concentration in urine samples.

FIGS. 6a-c show (a) a plot showing that a higher signal intensity and sensitivity is obtained by the IEF step; comparison between the EIEs (m/z 156.0±0.1) of spiked urine sample with a final added concentration of 16.0 μg/mL histidine after the application of the IEF step, and without the application of the IEF step; (b) integrated mass spectra of spiked urine sample with a final added concentration of 16.0 μg/mL histidine at the migration time (2.2 min) after application of the IEF step, a concentrated factor of 8.5 was achieved; and (c) integrated mass spectra at 2.2 min without application of the IEF step.

FIGS. 7a and b show before (a) and after (b) schematic representations of a separation carried out in an electroseparation syringe using acidic medium (such as 50 mM formic acid, pH 2.5) to positively charge the blood plasma proteins while the acidic compounds would be neutral, partially negatively charged or fully negatively charged based on their pKa values.

FIG. 8 shows a series of images demonstrating the focusing of 200.0 μg/mL of Chromeo™ 488 labelled human serum albumin (HSA) to the plunger as a cathode versus the constancy of a weak acidic dye (eosin B, 100.0 μg/mL) using a BGE composed of 50 mM formic acid (pH 2.5) in 30% (v/v) acetonitrile and applied voltage of −2000 V.

FIGS. 9a and b show (a) a chart of intensity over infusion time for naproxen (NAP) (8.0 μg/mL) and HSA (3.0 mg/mL) contained in the same mixture using the electroseparation syringe (ES) coupled with an electrospray ionisation-mass spectrometer analyser (ESI-MS) (the electroseparation step was accomplished by application of −2000 V for 640 seconds) (b) a plot of the average intensities of NAP and HSA versus the time of the ES step prior to ESI-MS analysis, average intensities of HSA and naproxen were obtained from the integrated mass spectra of the whole run “0-3.75 min”.

FIG. 10 shows calibration curves of average m/z peak intensity ratio of naproxen (●) and paracetamol (▪) using valproic acid as internal standard (IS) against concentration as described in Example 5.

FIGS. 11a and b show (a) EIEs (m/z 229.1, [M−1]⁻) of 16.0 μg/mL naproxen in (A) neat standard solution, (B) neat standard after application of the electroseparation step, (C) pre-spiked serum samples after application of the electroseparation step (the clean-up step), and (D) pre-spiked serum without the electroseparation step, where each sample is diluted by 15-fold in the aqueous solution; and (b) EIEs (m/z 150.2, [M−1]⁻) of 12.0 μg/mL paracetamol in (A) neat standard solution, (B) neat standard after application of the electroseparation step, (C) pre-spiked serum samples after application of the electroseparation step (the clean-up step), and (D) pre-spiked serum without the electroseparation step.

FIGS. 12a and b show mass spectra for (a) spiked serum sample after the electroseparation step; (b) spiked serum sample without the electroseparation step.

FIGS. 13a and b show before (a) and after (b) schematic representations of a separation carried out in an electroseparation syringe using basic medium (such as 300 mM ammonium hydroxide, pH 11.4) to negatively charge the serum proteins while the basic compounds would be neutral, partially positively charged or fully positively charged based on their pKa values.

FIG. 14 shows EIEs of m/z of 315>86, m/z 275>230, m/z 249>116, and m/z 267>190 for clomipramine (80.0 ng/mL), chlorphenamine (10.0 ng/mL), pindolol (50 ng/mL) and atenolol (250 ng/mL) in spiked serum samples, respectively, after the clean-up using the electroseparation syringe and without the clean-up step.

FIGS. 15a-d show the MS/MS spectra of clomipramine (80.0 ng/mL), chlorphenamine (10.0 ng/mL), pindolol (50 ng/mL) and atenolol (250 ng/mL) in spiked serum samples, respectively, after the clean-up step using the electroseparation syringe and without the clean-up step

FIGS. 16a and 16b are schematic illustrations of the components of two apparatus for performing the method of the present application, one based on a 6-port valve (FIG. 16a), and another based on a 8-port valve (FIG. 16b).

### DEFINITIONS

As used herein the term “net neutral analyte” or “net neutral compound” includes any compound having an overall neutral charge under the separation conditions employed. Accordingly, a net neutral molecule may be a neutral molecule, an amphiphilic molecule or a molecule that has an overall neutral charge under the electrochemical conditions present in the process of the invention.

The term “amphiphilic” in relation to a molecule is intended to mean a molecule comprising moieties of both positive and negative charge, such that the overall charge of the molecule is neutral. It will be appreciated that an amphiphilic analyte may be charged under some conditions (e.g. changed pH conditions). Amphiphilic analytes are sometimes referred to herein as ampholytes.

The term “syringe barrel” will be understood to broadly encompass an enclosed fluid passageway, which may be within a tubular structure or otherwise. The term “plunger” is used broadly to refer to a closure that is moveable within the syringe barrel. The plunger creates a closure within the syringe barrel, such that the syringe barrel has one open end and one closed end. The plunger may therefore be described as a moveable closure that can move from one end (a plunger receiving end) of the syringe barrel towards the open end to eject the liquid held in the syringe barrel, and away from the open end of the syringe barrel (towards the plunger receiving end) to draw liquids in through the open end of the syringe barrel.

The term “anode” is intended to refer to an electrode where oxidation occurs upon application of a voltage. The anode is within the electroseparation syringe.

The term “cathode” refers to the electrode where reduction occurs upon application of a voltage. The cathode is within the electroseparation syringe.

The term “electrode” refers to an electrode of any polarity, and includes grounded electrodes. A pair of electrodes may comprise a cathode and an anode, or a grounded electrode and a second electrode which is either an anode, a cathode, or switchable between an anode and a cathode.

The term “solution” is used broadly to refer to a solvent containing a compound (e.g. an analyte) in solution in the solvent. The solution may be described as a conducting solution. The solution in typical embodiments is an aqueous solution. The term “aqueous solution” includes any solution comprising water. Aqueous solutions may comprise additional suitable solvents and/or carriers, typically those that are miscible with water, such as polar solvents and/or carriers. Aqueous solutions and components thereof are described in further detail below.

The term compound is used to refer to a chemical substance other than solvents. Compounds may be analytes, being substances desired to be detected, and capable of being detected by the analytical techniques selected. In some embodiments the analyte is a substance that is not electrophoretically susceptible, and other compounds present in the sample are electrophoretically susceptible and constitute said compound (or compounds) that is/are re-distributed through the technique described herein. In other embodiments, it is the analyte that is the compound that is electrophoretically susceptible. Organic compounds are of particular interest as the compound that is subjected to re-distribution.

As used herein and in the appended claims, the singular forms “a,” “an,” and “the” include plural reference(s) unless the context clearly dictates otherwise. Thus, for example, a reference to “a compound” may include a plurality of compounds, and so forth.

The term “(s)” following a noun contemplates the singular or plural form, or both.

The term “and/or” can mean “and” or “or”.

Unless the context requires otherwise, all percentages referred to herein are percentages by weight of the composition.

Various features of the invention are described and/or claimed with reference to a certain value, or range of values. These values are intended to relate to the results of the various appropriate measurement techniques, and therefore should be interpreted as including a margin of error inherent in any particular measurement technique. Some of the values referred to herein are denoted by the term “about” to at least in part account for this variability. The term “about”, when used to describe a value, may mean an amount within 10%, 5%, 1% or ±0.1% of that value.

Unless defined otherwise, all technical and scientific terms used herein have the same meanings as commonly understood by one of ordinary skill in the art to which this invention belongs. It will be appreciated that any materials and methods similar or equivalent to those described herein can be used to practice or test the invention; the best-known embodiments of the various materials and methods are described.

The term “comprising” (or variations such as “comprise” or “comprises”) as used in this specification, except where the context requires otherwise due to express language or necessary implication, is used in an inclusive sense, i.e. to specify the presence of the stated features but not to preclude the presence or addition of further features in various embodiments of the invention.

### DESCRIPTION OF EMBODIMENT(S)

The invention provides a number of related processes that involve modifying the distribution of a compound such as an analyte in a solution.

One form of processes is for determining a concentration of an analyte in an aqueous solution, which comprises the analyte and a background electrolyte. The process comprises the steps of (a) applying a voltage across the aqueous solution in an electroseparation syringe, and (b) injecting the aqueous solution into an analyser.

The process for analysing an analyte in a solution (e.g. determining a concentration of the analyte) may comprise:
drawing the solution comprising the analyte into an electroseparation syringe comprising a syringe barrel and a plunger, and electrodes positioned to apply a voltage across a liquid contained in the syringe barrel,
applying a voltage across the solution in the syringe barrel to focus a concentration of the analyte within a region of the solution contained in the syringe barrel; and
injecting the solution into an analyser.

The plunger is operated to draw the solution into the electroseparation syringe, and to inject the solution from the electroseparation syringe and into the analyser.

The electroseparation syringe used in these processes comprises two electrodes, such as an anode and a cathode positioned to apply the voltage across the aqueous solution within the syringe barrel. In some embodiments, the processes may be used with existing microsyringes which comprise a metallic plunger and a metallic needle—although modification to the syringe may be required to remove or modify electrically insulating components within the syringe that are normally present to prevent or substantially prevent the flow of an electrical current across the contents within the syringe barrel. Embodiments of suitable electroseparation syringes are described below.

The inventors have surprisingly found that a stable neutralisation reaction boundary (NRB) can be formed using a lab-in-a-syringe approach, without the need for separated electrochemical chambers. The electroseparation syringe in embodiments of the invention accordingly comprises a single electrochemical chamber, or is free of separated electrochemical chambers.

The processes and systems of the invention may be used to focus a concentration of analyte within a region of the solution contained within the syringe. The analyte may be concentrated either inside or outside of the NRB. If the analyte is an amphiphilic molecule, it will typically be focussed within the NRB, whereas if the analyte is a charged molecule, it will be focussed in either the acidic or basic zones surrounding the NRB. Some analytes, such as acidic or basic molecules, may be charged under some conditions and be net neutral under other conditions, such as under different pH conditions. For example, a carboxylic acid will be net neutral under low pH conditions, and negatively charged when the pH of the aqueous solution exceeds the pKₐ of the acid. In the processes described herein, an analyte that can be either charged or net neutral under different conditions, may be focussed into different zones of the aqueous solution depending on the conditions (such as the pH) of the aqueous solution. Accordingly, in some embodiments, the step of applying the voltage across the solution may be referred to a focussing step, as the generation of an NRB causes the concentration of the analyte to be focussed into a region (or zone) of the solution within the electroseparation syringe.

In some embodiments, where the solution is an aqueous solution and comprises at least one additional compound (such as an interfering compound) in addition to the analyte and the background electrolyte, the application of the voltage across the aqueous solution is also a separation step. This separation involves an electrophoretic process within the electroseparation syringe. Therefore, the application of the voltage across the aqueous solution may cause separation in two-degrees; the first according to the charge of the molecules present in the aqueous solution, and the second according to pH of the molecules in solution.

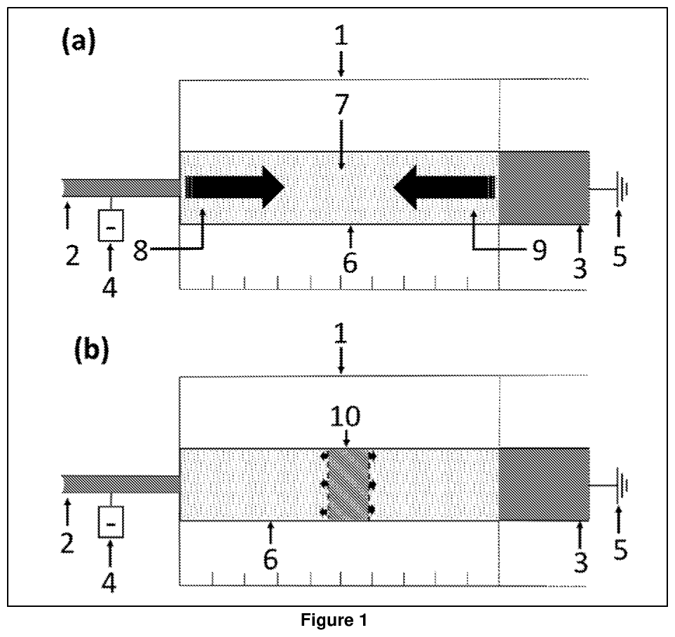

Application of the voltage across the aqueous solution causes water electrolysis at the anode and the cathode, creating a flux of H⁺ and OH⁻ within the electroseparation syringe. The inventors have shown (see, e.g., FIGS. 1, 3 and 4) that a neutralisation reaction boundary (NRB) is formed within the electroseparation syringe upon application of the voltage. Features of this NRB such as its position, length, direction, velocity, and the difference in pH across the interface-pH gap may be influenced by electric currents inside the system, sample nature and concentration, background electrolyte concentration, separation time, additives changing the viscosity, and solubility of analytes and/or contaminants in the aqueous solution. Accordingly, the processes may comprise adjusting the properties of the aqueous solution, the voltage applied to the solution, and the location of the anode and cathode, to control the features of the NRB. Through this NRB formation, the matrix effect can be reduced for analysis of complex samples, such as biological samples.

The processes cause the formation of the NRB within the barrel of the electroseparation syringe by electrolysing water contained in the aqueous solution at each electrode (FIG. 1), according to the following half-equations 1 and 2.

2H₂O₍ᵢ₎+2e⁻+H_{2(g)}+2OH⁻_{(aq)} Equation (1)

2H₂O₍ᵢ₎O_{2(g)}+4H⁺_{(aq)}+4e⁻ Equation (2)

Equation 1 will occur at the cathode generating OH⁻ ions and Equation 2 will occur at the anode generating H⁺ ions. This causes the generation of a zone of low pH at the cathode and a zone of high pH at the anode. Some of the OH⁻ ions generated at the cathode will migrate towards the anode, and some of the H⁺ ions generated at the anode will migrate towards the cathode. The NRB will form where the migrating H⁺ ions collide with the migrating OH⁻ ions, reacting to re-form water, i.e. the boundary where the migrating H⁺/OH⁻ ions meet, is where the neutralisation reaction occurs, hence it is called the NRB. There will therefore be a sharp change in pH at the NRB. In addition, the migrating H⁺ and OH⁻ ions also create a pH gradient across the distance from the electrode to the NRB, with the most extreme pH occurring at either electrode.

The voltage applied across the electrodes (e.g. the cathode and the anode) is therefore preferably sufficient to electrolyse water. The voltage may therefore be sufficient to establish a potential difference between the anode and the cathode of at least 1.23V; however typically the voltage will be sufficient to provide an overpotential for the electrolysis of water, i.e. the voltage difference between the electrodes will have a magnitude of greater than 1.23V. This may be achieved by supplying a voltage to one of the electrodes and connecting the other to ground, or it may be achieved by connecting both cathode and anode within a circuit. Since the voltage is applied to focus the concentration of a molecule in the aqueous solution, it is sometimes referred to herein as the focussing voltage.

In some embodiments, the magnitude of the focussing voltage will be from 1.23V to about 5000V. The sign of the voltage may be positive or negative. This may be achieved by establishing a voltage difference between the pair of electrodes, such as anode and cathode, from about −5000V to about 5000V, for example, from about −3000V to about 3000V, about −2500V to about 2500V or about −2200V to about 2200V, with the proviso that the focussing voltage excludes the range of −1.23V to +1.23V.

The focussing voltage is applied for a defined period of time, and in some embodiments, is stopped prior to the injecting step. Typically, the voltage is applied for up to about 1 hour, for example, up to about 45 minutes, about 30 minutes, about 25 minutes, about 20 minutes, about 15 minutes, about 10 minutes, about 5 minutes, about 2 minutes, about 1 minute or less. The voltage will typically be applied for at least about 1 second, at least about 5 seconds, at least about 10 seconds, at least about 15 seconds, at least about 20 seconds, at least about 30 seconds at least about 1 minute, or at least about 5 minutes. Any of these minimum times may be combined with any of the above maximum times to form a range, provided the minimum time is less than the maximum time. The duration of the focussing voltage application may, for example, be between 5 seconds and 10 minutes, 5 seconds and 5 minutes, 5 seconds and 2 minutes, 10 seconds and 10 minutes, and so forth.

Following the focussing step, the solution is injected (or infused) into an analyser. In some embodiments, a portion of the solution is injected into the analyser, wherein the portion comprises a region of the solution comprising the analyte. In some embodiments, the region comprising increased concentration of the analyte is the NRB. In other embodiments, the region comprising increase concentration of the analyte is not the NRB, for example, where the analyte is charged and the NRB contains interfering amphiphilic or neutral compounds. In some embodiments, a portion of the solution not containing the analyte is discarded.

In some embodiments, the solution is expelled from the syringe in portions, often separated by a defined period of time. The portions may be defined by a volume of solution, for example, each portion may be 1-5 μl in volume, or each portion may correspond to the acidic, NRB and/or basic zones of the electrolysed solution. In some embodiments, the portions may be injected or infused into the analyser, discarded as waste, or collected as a fraction. The defined period of time is typically sufficient to allow discrete analysis of the contents of each portion of the solution and may therefore depend on the analyser selected. In some embodiments, the period of time is about 1 minute to about 30 minutes.

The total volume of solution that is drawn into the syringe barrel (and then injected into the analyser) may be between 0.5 pl and 1 ml, and preferably between 0.5 μl and 20 μl. The volume may be between 0.5 μl and 10 μl, or between 0.5-5 μl, or between 1 and 5 μl.

In some embodiments, the solution is injected into a flow of a sheath liquid. Any suitable sheath liquid that is compatible with the analysis technique may be used. The sheath liquid may comprise the same background electrolyte as the solution (discussed below).

Any analyser capable of receiving the solution from the electroseparation syringe and analysing the analyte may be used. Suitable analysers include mass spectrometers (MS), UV-visible spectrophotometers, infrared spectrometers, ramen spectrometers, or any of these coupled with a further technique, such as high-performance liquid chromatography, gas chromatography and electrospray ionisation (ESI) or a combination thereof. Preferably, the analyser is a mass spectrometer. Any type of mass spectrometer may be used; however, ESI-MS is a convenient analysis technique for the present processes. The analyser may be adapted for on-line analysis.

In some embodiments, a voltage is applied to the solution during the injection step. This voltage (sometimes referred to herein as the infusion voltage) may be of the same sign and magnitude as the focussing voltage. When the application of the focussing voltage to the solution is stopped, the molecules in solution will tend to disperse. If enough time elapses (typically hours), the solution will return to equilibrium. Thus, when the voltage is stopped, the concentration of the net neutral molecules in the NRB will disperse through the solution, and the charged molecules diffuse away from the electrodes. Accordingly, maintaining a voltage during the injection step may assist in maintaining the NRB and provide a more focussed concentration of the net neutral molecules within a region of the solution.

In some embodiments, the infusion voltage difference between the anode and cathode will be from about −5000V to about 5000V, for example, from about −3000V to about 3000V, about −2500V to about 2500V or about −2200V to about 2200V.

In some embodiments, the focussing voltage is applied under increased pressure. The pressure increase may be achieved by the inclusion of a valve at the discharge end of the barrel of the electroseparation syringe. Hydrogen and oxygen gas are released during water electrolysis at the anode and the cathode, which when the valve is closed cause a pressure build-up within the syringe. In addition, the plunger may be depressed with the valve closed, for example by advancing a syringe pump or by hand, to increase the pressure of the solution (e.g. the aqueous solution) within the barrel of the electroseparation syringe. Surprisingly, the processes carried out under increased pressure demonstrate improved analytical results. Without wishing to be bound by theory, the improved results are believed to be achieved by the critical suppression of bubble formation inside the syringe barrel during the electroseparation step due to the enhanced solubility of the generated gases under increased pressure.

The processes may be used to determine the concentration of an analyte of interest, or may be used to analyse solutions to determine the identity of an analyte in the solution. Typically, the identity of the analyte is known prior to detection and it is the concentration that is desired to be determined by analysis.

The processes may further comprise a step of drawing (or aspirating) the solution into the electroseparation syringe. Using the example of an aqueous solution as the test solution, the aqueous solution may be aspirated into the syringe by itself, or between different solutions. Aspiration of the aqueous solution before and/or after a different solution may, for example, be used to adjust the pH, introduce an internal standard, or perform a chemical modification (e.g. alkylation, acetylation, esterification or other transformation of an acidic or basic moiety) on the analyte or other molecule contained in the aqueous solution, such as an interfering protein. This drawing step may comprise drawing an aliquot of the aqueous solution into the electroseparation syringe. Knowing the volume of aqueous solution in the electroseparation syringe assists in the calculation of analyte concentration. The use of an electroseparation syringe comprising volumetric indicia assists in determining the volume of the aliquot drawn into the syringe. Alternatively, the syringe pump may be operated by a control system that is able to calculate the volume of solution (including the test solution, and optionally different solutions before and/or after the test solution) being drawn into the syringe barrel, in which case volumetric indicia may not be required.

The concentration of an analyte in an aliquot (i.e. a defined volume) of the solution may be quantified by comparison with a concentration curve for the subject analyte using the analyser selected. Determining the amount of analyte present in a known volume of the solution allows calculation of the concentration of analyte in the bulk solution (e.g. bulk aqueous solution).

Also provided herein is a process for focussing a concentration of a molecule in a solution such as an aqueous solution, comprising applying a voltage across the solution comprising the molecule and a background electrolyte in an electroseparation syringe to generate a region comprising an increased concentration of the molecule in the solution, the electroseparation syringe comprising a pair of electrodes, e.g. an anode and a cathode, positioned to apply the voltage across the solution. In some embodiments involving an aqueous solution, the molecule is an amphiphilic molecule which is concentrated into a region of the aqueous solution corresponding with an NRB. In some embodiments, the molecule is a charged molecule which is concentrated into a region of the aqueous solution that is not the NRB. In some embodiments, the molecule is an analyte which is injected from the electroseparation syringe into an analyser.

Any of the solutions (e.g. aqueous solutions), background electrolytes, voltages and electroseparation syringes described herein may be used in this process. This process may further comprise any of the steps of the other processes described herein. Accordingly, the steps described above for those processes for determining a concentration of analyte in a solution apply equally to processes that are designed more generally for focussing the concentration of the compound/molecule in the solution.

Also provided herein is a process for separating a charged compound from an amphiphilic compound, the process comprising applying a voltage across a solution (e.g. an aqueous solution) comprising the charged compound, the amphiphilic compound and a background electrolyte in an electroseparation syringe. The electroseparation syringe comprises a pair of electrodes, which may be an anode and a cathode, positioned to apply the voltage across the aqueous solution. In some embodiments, this process further comprises isolation of the charged and/or amphiphilic compound(s) following their separation. In some embodiments, the charged and/or amphiphilic compound(s) are isolated following subjection to a further separation step, such as high-performance liquid chromatography (HPLC).

Any of the solutions (e.g. aqueous solutions), background electrolytes, voltages and electroseparation syringes described herein may be used in this process. This process may further comprise any of the steps of the other processes described herein. Accordingly, the steps described above for those processes for determining a concentration of analyte in a solution apply equally to processes that are designed for separating charged compounds from amphiphilic compounds in a solution. The separation is initially into different regions of the solution within the syringe barrel, and then those regions of the solution can be separated into two physically separate samples (i.e. isolated).

### Solution(s)

The processes of the invention require the application of a voltage across the solution. The solution is a conductive solution. The solution suitably comprises the solvent and an electrolyte. In the case of an aqueous solution, the aqueous solution comprises water and a background electrolyte.

In some embodiments, the aqueous solution comprises a biological sample, such as a blood, urine, hair, faecal or tissue sample. Typically, the biological sample is optionally treated and then diluted with the other components of the aqueous solution described below. The compounds requiring analysis may therefore be biological compounds.

The aqueous solution comprises a sufficient concentration of water for electrolysis to occur at the electrodes. The aqueous solution may comprise at least about 10 vol % water, for example, at least about 15 vol %, about 20 vol %, about 25 vol %, about 30 vol %, about 35 vol %, about 40 vol %, about 45 vol %, about 50 vol %, about 55 vol %, about 60 vol %, about 70 vol %, about 80 vol %, about 90 vol %, about 95 vol %, about 99 vol % or about 99 vol %. In some embodiments, the aqueous solution is substantially free of non-water solvents.

In some embodiments, the aqueous solution comprises a solvent (i.e. an organic solvent) in addition to water. The solvent is preferably miscible with water. Suitable solvents include acetonitrile (ACN), dimethylformamide (DMF), methanol (MeOH), ethanol (EtOH), or a combination thereof. In some embodiments, the aqueous solution comprises a polar solvent in an amount of up to about 90 vol %, for example, up to about 80 vol %, about 70 vol %, about 60 vol %, about 50 vol %, about 40 vol % or about 30 vol %.

The aqueous solution also comprises a background electrolyte.

The background electrolyte acts to balance the charge and ion flow of the H⁺/OH-flux caused by the electrophoresis of water at the anode and cathode. Preferably, the background electrolyte does not interfere with the analyser, or put another way is not detected by the analyser.

The background electrolyte may be included in the aqueous solution in a concentration of about 0.01 mM to about 1000 mM, for example, from about 0.05 mM to about 250 mM or about 0.1 mM to about 150 mM.

The background electrolyte is typically an ionic species. When used in an analytical method, any ionic species compatible with the selected analyser may be used. For example, for mass spectrometer analysers, suitable classes of background electrolyte include an ammonium salt, a carboxylic acid, a carbonate, a carbamate, a thiocarbonate (including mono-, di- and tri-thiocarbonates), a borate salt, a carboxylate salt and an amine, or a combination thereof. Some examples of specific background electrolyte species include ammonium acetate, formic acid, ammonium hydroxide, acetic acid, sodium acetate, potassium acetate, sodium formate, potassium formate, sodium carbonate and calcium carbonate, sodium phosphate, potassium phosphate, ammonium phosphate, triethylamine or a combination thereof. For a fluorescent analyser any ionic species that is non-fluorescent may be used, including all of the suitable background electrolytes suitable for mass spectrometry analysis described above and also sodium salts, potassium salts, calcium salts, magnesium salts, chloride salts, sulfate salts, phosphate salts, and so on.

In the analytical processes described herein, the aqueous solution comprises an analyte. The analyte will typically be a net neutral analyte. Accordingly, the analyte may be a neutral analyte or an amphoteric analyte. However, in some embodiments, the analyte may be a charged (either cationic or anionic) molecule.

Neutral analytes include compounds unable to carry a charge, or compounds that would not carry a charge under the conditions present in the aqueous solution. For example, neutral analytes include weak acids and bases. Weak acid analytes include carboxylic acids, carbamates, carbonates, thiocarbonates, thiocarboxylic acids, alcohols, phenols, and thiols, or a combination thereof. Weak base analytes include amines (primary, secondary and tertiary).

When the analyte is a net neutral analyte, the application of the voltage across the aqueous solution causes focusing of the analyte concentration into a region of the aqueous solution (e.g. within an NRB).

When the analyte is a charged species, the application of the voltage causes focussing of the analyte concentration near one of the anode or the cathode, depending on whether the analyte is positively or negatively charged. If any interfering net neutral molecules are present in the aqueous solutions they may also be focused within a region of the solution (e.g. an NRB).

When the analyte is a weak acid, the aqueous solution preferably has a pH lower than the pKa of the analyte. The pH of the aqueous solution may therefore be adjusted by addition of an acid to ensure that the weak acid remains in an uncharged form during the separation step. Further, the acid added to the aqueous solution is preferably not detected by the analyser. For example, in processes employing analysis by mass spectrometry, the pH of the aqueous solution may be adjusted by addition of an acid selected from formic acid, acetic acid, and carbonic acid, or a combination thereof. Typically, the aqueous solution comprises about 0.2-5 vol % of the acid. The amount may be around 0.5-2 vol %, for example about 1 vol % of the acid.

When the analyte is a weak base, the aqueous solution preferably has a pH higher than the pKa of the analyte. The pH of the aqueous solution may therefore be adjusted by addition of a base to ensure that the weak base remains in an uncharged form during the separation step. Further, the base added to the aqueous solution is preferably not detected by the analyser. For example, in processes employing analysis by mass spectrometry, the pH of the aqueous solution may be adjusted by addition of a base selected from ammonia (NH₄OH), methylamine, triethylamine, N,N-diisopropylethylamine, pyridine, aniline, pyrrolidine, N-methylpyrolidine, an inorganic hydroxide salt (e.g. sodium hydroxide, potassium hydroxide, calcium hydroxide and so on), an inorganic carbonate (e.g. sodium carbonate, potassium carbonate, calcium carbonate and so on) or a combination thereof. Typically, the aqueous solution comprises about 0.2-5 vol % of a liquid base or a 0.2-5 wt % solution of a solid base. The amount may be around 0.5-2 vol %, for example about 1 vol % of a liquid base or a solution of a solid base (e.g. a 1M solution of a solid base, such as an inorganic hydroxide or carbonate), or 0.5-2 wt %, e.g. 1 wt % of a solid base.

In some embodiments, the analyte is an amphoteric analyte. Amphoteric analytes include any molecule comprising one or more weakly acidic moieties and one or more weakly basic moieties, including proteins, peptides and amino acids, and combinations thereof. In some embodiments, an amphoteric analyte is a net neutral analyte, while in others the amphoteric analyte may be a charged analyte, depending on the aqueous solution conditions.

The aqueous solution, may comprise one or more additional components. For example, the aqueous solution may comprise a reference analyte, a surfactant, a rheology modifier, an indicator, a chaotropic agent, an oxidizing agent, and a reducing agent or a combination thereof.

A reference analyte may be included in the solution (e.g. aqueous solution) for calibration purposes. Any net neutral molecule may be used as reference analyte. The reference analyte is added to the aqueous solution in a predetermined concentration. Typically, prior to its selection as reference analyte, a calibration curve for the reference analyte will be prepared. In some embodiments, the reference analyte may be included in the aqueous solution in a concentration of about 1 g/ml to about 100 g/ml. The reference analyte may be of the same class of analyte (e.g. an ampholyte with similar pl) as the analyte.

The aqueous solution may further comprise a surfactant. The surfactant may prevent agglomeration of particulates in the syringe and assist solubilise less soluble components in a complex sample. Any suitable surfactant that is compatible with electrolysis and the selected analysis technique may be used. The surfactant may be selected from a non-ionic, cationic or anionic surfactant. Suitable examples of surfactants include sodium dodecyl sulfate, cetyltrimethlammonium bromide, ethoxylated sorbate (e.g. Tween 20 and Tween 80), ethoxylated phenol (e.g. Triton X), or a combination thereof. Typically, the surfactant may be included in a concentration of 0.01 g/ml to 1000 mg/ml.

The aqueous solution may further comprise a rheology modifier. The rheology modifier alters the viscosity and flow properties of the aqueous solution. Any suitable rheology modifier that is compatible with electrolysis and the selected analysis technique may be used.

The aqueous solution may further comprise an indicator. Any suitable indicator that is compatible with electrolysis and the selected analysis technique may be used. For example, the indicator may be a pH indicator. The addition of a pH indicator can be helpful to observe NRB formation. In some embodiments, an indicator is included in a first run of the process to assist in optimisation of conditions, and may be omitted from later runs of the process.

Chaotropic compounds disrupt the hydrogen bonds and hydrophobic interactions both between and within proteins. They can enhance the proteins solubilization if they are used in suitable concentration. Suitable chaotropic compounds include urea, substituted urea, and guanidinium salts.

The aqueous solution may comprise an oxidizing or reducing agent to control the electrolysis process, e.g., to be reduced or oxidized before the water or the target analyte. Examples of reducing agent include ascorbic acid, thiosulphates and reducing sugars. Examples of oxidizing agents include peroxides (such as hydrogen peroxide and alkyl peroxides) and quinones.

It may be convenient to provide a pre-mixed aqueous solution to be used as diluent for a sample, such as a biological sample. This pre-mixed aqueous solution may comprise any of the above described components of the aqueous solution in a pre-determined amount. For example, in one embodiment, there is provided a solution comprising: (i) a predetermined amount of a background electrolyte, (ii) a predetermined amount of a reference analyte; and (iii) a predetermined volume of solvent. The solvent may be water, or may be any of the other solvents described above. When the solution does not comprise water, water will be added to form the aqueous solution as used in the processes of the invention.

Also provided herein is a kit of reagents (parts) comprising: (i) a predetermined amount of a background electrolyte, (ii) a predetermined amount of a reference analyte; and optionally (iii) a predetermined volume of solvent.

Also provided herein is a kit of reagents (parts) comprising: (i) a combination of a predetermined amount of a background electrolyte and a predetermined amount of a reference analyte; and (ii) a predetermined volume of solvent.

The reagent kit may further comprise one or more of the above described surfactants, rheology modifiers, indicators, chaotropic agents, reducing compounds and oxidising compounds combined in any of the separate parts of the kits described herein, or in a further separate part.

### Electroseparation Syringe

The processes described herein may employ, as the electroseparation syringe, a commercial microsyringe comprising a conductive needle and conductive plunger. Commercial microsyringes typically comprise a metallic needle, a syringe barrel (which is typically a glass barrel) and metal plunger. As described in the following passage, such microsyringes usually further comprise an electrically insulating fitting that prevents contact between the metal plunger and liquid contents held within the syringe barrel, which may require modification to be suited to use in the applications described herein.

The plunger of conventional commercial microsyringes typically comprise a non-conductive fitting on the solution facing end of the plunger (herein referred to as the plunger head) to form a seal with the internal wall of the barrel when the plunger is in place. The fitting may be formed of an electrically insulating material, such as plastic or rubber-like material. This fitting typically extends around the plunger head, providing a sliding surface between the metal plunger head and the internal surface of the syringe barrel within which the plunger head slides. The fitting fits snug within the syringe barrel to ensure no leakage. The fitting also provides electrical insulation to prevent any unintentional completion of an electrical circuit between the metal needle and the plunger, via the liquid drawing into the syringe barrel. To adapt such conventional microsyringes for use in the present application, the syringe must be configured or adjusted to provide electrical contact, in use, between the liquid contents of the syringe barrel and either the metal plunger (where a metal plunger is present) or the electrode at the plunger end of the electroseparation syringe. This can be achieved by modifying the fitting to contain an opening to allow contact between the plunger head and any liquid when drawn into the syringe body, or replacing the fitting with a modified fitting that allows such contact.

Commercial microsyringes also do not comprise power supply connectors, which are necessary for the application of a voltage to the aqueous solution as required by the processes described herein.

The present application provides an electroseparation syringe comprising a syringe barrel, a plunger, and a pair of electrodes, wherein the electrodes are configured to come into electrical contact with a solution contained within the barrel in use, so as to enable a voltage to be applied longitudinally across solution contained in the barrel.

The term “plunger” is used broadly to refer to a closure that is moveable within the syringe barrel. The plunger creates a closure at one end of the syringe barrel, such that the syringe barrel has one open end and one closed end. The plunger may therefore be described as a moveable closure that can move from one end (a plunger receiving end—noting that the closed end may be only part-way towards the plunger-receiving end) of the syringe barrel towards the open end to eject the liquid held in the syringe barrel, and away from the open end of the syringe barrel (towards the plunger receiving end) to draw liquids in through the open end of the syringe barrel. The plunger is slideable in a water-tight engagement with the inner walls of the syringe barrel. Through movement of the plunger within the syringe barrel the plunger can hydrodynamically draw liquids into the syringe barrel or expel liquids from the syringe barrel.

The electrodes preferably each comprise a power supply connector for connection to a power supply, that in use enables the application of a voltage across solution contained in the barrel. The pair of electrodes may comprise a cathode and an anode. The electrodes may alternatively be a cathode and a grounded electrode, or a grounded electrode and an anode. It is possible for the polarity of the electrode(s) to be changed according to the voltage potential applied (positive or negative).

The electrodes may each be provided by features of the syringe or by a needle fitted to the syringe, as described in further detail below.

It will be understood that the electroseparation syringe of the present application may be supplied as a complete unit, or it may be supplied as a kit of parts that includes some or all of the electroseparation syringe features specified herein. Thus, in one example, the electroseparation syringe may be supplied without one of the electrodes (e.g. when one electrode is constituted by a needle), and the needle can be supplied separately.

In another embodiment, the electroseparation syringe comprises:
a barrel (i.e. a syringe barrel) having a discharge end and a receiving end (i.e. an end that receives the plunger);
a plunger;
a cathode comprising a first power supply connector; and
an anode comprising a second power supply connector,
   wherein the cathode and the anode are configured to provide a voltage across a solution contained in the barrel. The electroseparation syringe may further comprise an opening (which may be described as an outlet) at the discharge end of the barrel. The opening may comprise a needle. The needle may be connected directly to the discharge end of the barrel or it may be connected to the discharge end through a needle assembly.

Each of the electrodes (e.g. the anode and the cathode, but similarly the grounded electrode) may comprise or consist of any suitable electrode material. Each electrode may be integrated into a part of the electroseparation syringe (e.g. the barrel, plunger, needle or needle assembly) or it may be an additional component attached or embedded within a part of the electroseparation syringe. In some embodiments, an entire part of the electroseparation syringe (such as the plunger or the needle) consists of a conductive material and therefore the entire part may be considered the anode or the cathode. Accordingly, in one embodiment, one of the electrodes is constituted by the plunger. Alternatively, the electrode at the end of the syringe that receives the plunger may be provided by a conductive fitting that forms part of the plunger, or is positioned so as to come into contact with liquid held within the syringe barrel (in use) at the receiving end of the syringe. In one embodiment, one of the electrodes is constituted by the needle. The electroseparation syringe may be supplied in combined form with a needle, or it may comprise a needle receiver at a discharge end of the syringe, adapted to receive a needle. In a variation on this embodiment, the electrode at the needle end of the electroseparation syringe may be provided by a metal fitting at the discharge end of the syringe barrel. The metal fitting may be shaped to receive a needle, or otherwise. To constitute the required electrode, the metal fitting needs to come into contact with liquid contained within the syringe barrel, in use. In yet another variation, the electrode at the end of the syringe barrel through which liquids are drawn into and discharged from the syringe may be in the form of a metal plate, disc, coating, rod or fitting of any other shape that sits inside the syringe barrel at the discharge end. In the case of metal plates/discs, electrode may contain a central aperture through which the liquids are drawn into the syringe.

The term “needle” is used broadly to refer to an elongate tube, which is typically metal in the embodiments described herein, with a central bore. Sharpness is not to be read as being a feature required for a metal tube to constitute a needle.

In view of the conventional use of metal needles and metal plungers in conventional microsyringes, in some embodiments the plunger will constitute one electrode and the needle supplied with or fitted to the electroseparation syringe will constitute the other of the electrodes.

Whether the conductive material is the anode or the cathode, or a grounded electrode, will depend on how each electrode is connected to the power supply, as the power supply supplies electrons to the cathode to drive the reduction of water. Accordingly, in any embodiment of the electroseparation syringe described herein, the location of the anode and cathode may be swapped. In some embodiments, when a negative voltage is applied to the cathode through the second power supply connector, the first power supply connector may connect the anode to ground. Alternatively, when a positive voltage is applied to the anode through the first power supply connector, the second power supply connector may connect the cathode to ground.

Each of the anode and the cathode comprises a power supply connector. The cathode comprising the first power supply connector and the anode comprising the second power supply connector. The power supply connectors include any means of connecting the anode and/or cathode to a power supply. The first and second power supply connectors may be the same or different. In some embodiments, the first and/or second power supply connector may be a wire (or lead) extending from a power supply to the respective cathode and/or anode. In other embodiments, the first and/or second power supply connector may be a portion of the electrode adapted to interface with the power supply, for example, the power supply connectors may comprise a conductive extension from the anode and/or cathode shaped to connect to a power supply, for example, by connection with a wire or a lead.

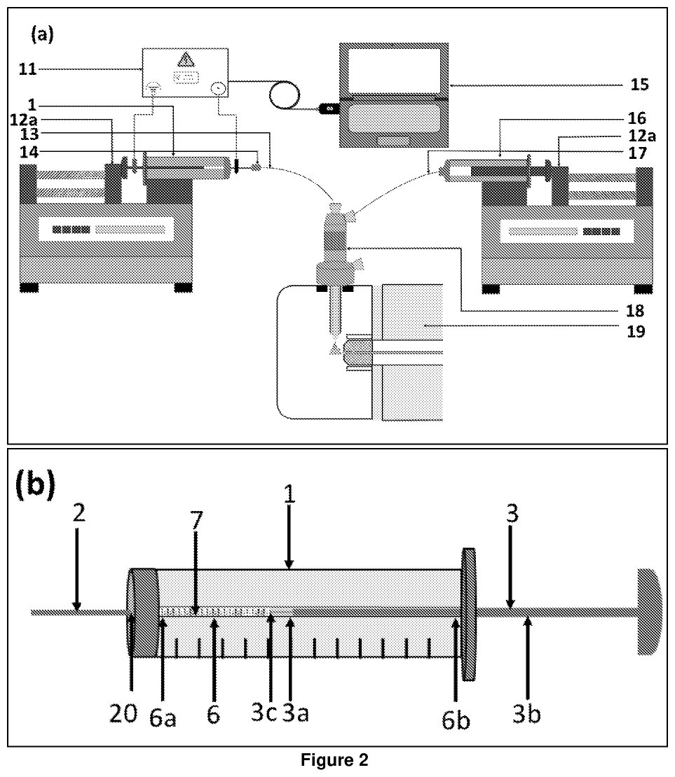

The power supply will provide direct current (DC) electrical power. Any suitable DC power supply may be used. For example, the power supply may be a USB power supply as shown in FIG. 2. The power supply may be a high voltage power supply.

In some embodiments, one of the cathode or the anode is positioned at the discharge end of the barrel. For example, the anode may be comprised within a needle or a needle assembly.

In some embodiments, the plunger may comprise one of the cathode or the anode. When the plunger consists of a conductive material, such as a metal, the entire plunger may provide the anode or the cathode. Alternatively, the plunger may comprise a conductive portion positioned in a solution-contacting portion of the plunger which constitutes the electrode portion. The plunger may alternatively comprise a grounded electrode.

In some embodiments, the anode or the cathode may be positioned within the barrel of the electroseparation syringe. The barrel may therefore comprise an electrode at or near the discharge end, and/or positioned within the barrel of the electroseparation syringe at a location spaced apart from the discharge end of the barrel towards the receiving end of the barrel. The two electrodes will be positioned within the electroseparation syringe such that when a solution is contained within the barrel of the syringe and a voltage is applied across the electrodes, the voltage will travel through the solution. Accordingly, the anode and the cathode will not be positioned such that they will touch when a solution is contained in the barrel of the syringe, i.e. when the plunger is withdrawn from the barrel towards the receiving end. The anode and the cathode may contact each other when no solution is contained in the syringe, such as when the plunger is completely depressed towards the discharge end of the barrel.

In some embodiments, the syringe barrel may comprise an internal coating. The internal coating may cover the entire internal wall of the syringe barrel, or it may cover a portion of the internal wall that will contact a solution drawn into the syringe barrel. The internal coating may comprise a non-conductive and/or chemically inert material. For example, the internal coating may comprise a film-forming polymer or copolymer, such as a cellulose ether (e.g. hydroxypropyl methylcellulose, ethylcellulose, Cellulose acetate phthalate), an acrylic polymer (e.g. polymethacrylate and methacrylate aminoester copolymer), a polyethylene glycol, a polyvinyl pyrrolidone, a polyvinyl alcohol and a wax, or a combination or copolymer thereof. The internal coating may be applied by contacting a solution comprising the film-forming polymer or copolymer and a solvent with the internal wall of the barrel and removing the solvent.

The capacity of the syringe barrel (i.e. the maximum volume that may be taken up into the syringe barrel) may be between 0.5 μl and 1 ml. The capacity is preferably a minimum of 1 μl, 2 μl, 3 μl, 4 μl or 5 μl. The capacity is preferably not more than 100 μl, 50 μl, 20 μl, 15 μl or 10 μl. Any maximum and minimum capacity can be combined to form a range, such as a capacity range of 1 μl-15 μl. The fact that the separation or re-distribution of the compound/analyte within this volume of liquid is performed using electrophoretic techniques in a syringe (prior to the sample being injected into other analytical equipment for formal analysis), is a notable aspect of the present invention. It is noted that the larger the volume of liquid, the higher the heat generation, which has caused those skilled in the art to tend towards a lower voltage potential when performing the electrophoretic separation to avoid excessive heat. In spite of this potential impediment, the applicants use this volume of liquid in the syringe barrel and perform the separation with a high voltage potential. Through performing this type of pre-treatment step in the syringe, applicants have achieved an improvement in the overall analytical process. It is a unique feature of the present invention that a pre-treatment is performed in the syringe itself as a form of pre-analysis resolution step, prior to performing a complete analytical measurement. It is a further unique feature of preferred embodiments that the pre-treatment step is performed under a pressure that suppresses bubble formation, which adds to the improvement in the analytical results achieved. Through this pre-treatment step, a surprising improvement is made in the overall analytical results as compared to the analysis performed without this pre-treatment step.

In some embodiments, the electroseparation syringe further comprises a valve associated with the discharge end of the barrel. The valve may be located at the discharge end of the syringe barrel. The valve may alternatively be in fluid connection to the discharge end of the syringe barrel via a fluid passageway. Any valve suitable for controlling the release of a liquid and/or a gas from the discharge end of the electroseparation syringe may be used. The valve enables the pressure of the aqueous solution to be increased when the focussing voltage is applied. Surprisingly, it has been found that the electrolysis step and NRB formation is superior when the pressure of the aqueous solution is increased.

Also provided herein is an electroseparation syringe comprising a barrel having a discharge end and a plunger end (or plunger receiving end); a plunger; a cathode; an anode and a valve located at the discharge end of the barrel, wherein the cathode and the anode are configured to provide a voltage across a solution contained in the barrel.

The electroseparation syringe may comprise volumetric indicia, for example markings or etchings along the exterior wall of the barrel. While the analytical processes described herein may be used to provide qualitative analysis of an analyte, in order to provide quantitative data regarding the concentration of the analyte in the aqueous solution, the volume of solution analysed must be known. The inclusion of volumetric indicia on the electroseparation syringe allows for ready determination of the volume of aqueous solution subjected to the processes of the invention.

Any of the electroseparation syringes described herein may be used in the analytical and/or separatory processes of the invention.

One embodiment of an electroseparation syringe is illustrated in FIG. 2b. This electroseparation syringe may be incorporated into the system of FIG. 2a, discussed in detail below.

Across all Figures, the following reference numbers are used:

| | |
|---|---|
| 1 | Electroseparation syringe |
| 2 | Needle |
| 3 | Plunger |
| 3a | Plunger head |
| 3b | Central shaft of the plunger |
| 3c | Exposed area of the plunger head |
| 4 | Cathode |
| 5 | Ground |
| 6 | Syringe barrel |
| 6a | Discharge end of the syringe barrel |
| 6b | Receiving end of the syringe barrel |
| 7 | Solution-sample diluted with the background electrolyte |
| 8 | Flux of hydroxyl ions |
| 9 | Flux of protons |
| 10 | Neutralization reaction boundary (NRB) where |
| | the amphoteric analytes can be focused |
| 11 | High voltage power supply |
| 12a | Standard syringe pump |
| 12b | Syringe pump with built-in points (connections) |
| | for high voltage application |
| 13 | Capillary tubing (ID 50 pm, length 25 mm, volume 0.5 μL) |
| 14 | Fitting to connect the needle to the capillary tubing |
| 15 | Computer controlling the high voltage power supply |
| 16 | Sheath liquid infusion |
| 17 | Peek tubing |
| 18 | Sprayer |
| 19 | Mass spectrometer |
| 20 | Needle fitting |
| 21 | Anode |
| 22a | 6-port valve |
| 22b | 8-port valve |
| 23 | Controller |
| 24 | Button to reverse the polarity |
| 25 | Input voltage module |

The electroseparation syringe (1) comprises a syringe barrel (6), a plunger (3) and a needle (2). Solution (7) is drawn into the syringe barrel through the needle (2) at the discharge end (6a) of the syringe barrel (6). The plunger is positioned at the receiving end (6b) of the syringe barrel. The plunger (3) comprises a plunger head (3a) and a central shaft (3b). The plunger head comprises anon-conductive resilient material fitting around its periphery that comes into contact with the inner wall of the syringe barrel (6) to prevent leakage of the solution (7). There is an exposed area of the plunger head (3c) of the plunger (3) that constitutes one electrode, and the needle (2) constitutes a second electrode—one a cathode, the other an anode—that apply a voltage across the solution (7) longitudinally within the syringe barrel (6). The needle is connected to the syringe barrel via a needle fitting (20). In this vicinity there is also a valve (not illustrated in detail) that can be opened and closed so as to increase the pressure on the solution when it is expelled from the syringe barrel (6). The syringe barrel (6) has an internal coating (not shown), and indicia marked to show the volume of solution in the syringe barrel.

### Systems

Also provided herein is an analytical system, comprising:
an electroseparation syringe comprising a syringe barrel, a plunger and a pair of electrodes (e.g. an anode and a cathode) positioned to apply a voltage through an aqueous solution contained in the syringe barrel in use;
a power supply configured to connect with the anode and the cathode; and
an analyser adapted to receive and analyse an analyte from the electroseparation syringe.

The electroseparation syringe may be any electroseparation syringe described herein. When the electroseparation syringe comprises the first and second power supply connectors, the power supply may connect with the anode and the cathode through the connectors.

One embodiment of this system is shown in FIG. 2a. The electroseparation syringe (1) is connected to power supply, such as power source (11), through two power supply connectors; the first power supply connector extending between the power source (11) and one electrode of the electroseparation syringe (which could be a cathode, but is marked as an anode in FIG. 2a) and the second power supply connector extends between the power source (11) and the anode of the electroseparation syringe (1). The power source (11) is connected to a controller through a USB connection. As shown in FIG. 2a, the controller is a laptop physically connected to the power supply; however, in other embodiments, the controller may be remote from the power supply interfaced using a wireless technology, such as IR, Wi-Fi or Bluetooth.

The electroseparation syringe is connected to syringe pump (12a) which can be programmed to inject a solution contained in the syringe (1) at a desired time and at a desired rate. In some embodiments, a syringe pump is also controlled by a controller (15), which may be the same controller (15) as for the power supply. The needle at the discharge end of the syringe (1) is connected to a receiver or fitting (14). The receiver/fitting comprises an inlet to receive a solution from the syringe (1) and a connector (15) to supply the received solution to the analyser (7, 8). The connector (15) shown in FIG. 2a is capillary tubing having an internal diameter of 50.0 m; however, any suitable connector may be used.

As shown in FIG. 2a, the analyser is an ESI-MS (19); however, in other embodiments, the analyser may be any of those described herein. The sample is sprayed by sprayer (18) into the mass spectrometer (19). Also shown in FIG. 2a, is a second syringe for sheath liquid infusion (16) connected to a second syringe pump (12a). The second syringe (16) is employed to provide a sheath liquid into the analyser at a rate controlled by the second syringe pump. In some embodiments, a second syringe pump may be controlled by a controller, which may be the same controller as for the power supply.

Also provided herein is a system, comprising:
a receiver for an aqueous solution injected from an electroseparation syringe comprising an anode and a cathode positioned to apply a voltage through the aqueous solution when contained in the electroseparation syringe, and
a power supply configured to connect with the anode and the cathode.

The electroseparation syringe may be any electroseparation syringe described herein. When the electroseparation syringe comprises the first and second power supply connectors, the power supply may connect with the anode and the cathode through the connectors.

An embodiment of this system is also shown in FIG. 2a as a sub-system of the overall analytical system shown. The embodiment of this system comprises a receiver and power source (11). The receiver may comprise an inlet (provided as microtight fitting (14)) and a connector (provided as capillary tubing (13)). The receiver may be adapted to form a liquid tight seal when engaged with the needle of the syringe and supply the received aqueous solution to an analyser. In some embodiments, the receiver of this system may comprise only a portion of tubing (13) in addition to fitting (14), or may comprise only a fitting for the electroseparation syringe suitable for interfacing with the analyser. Alternatively, the receiver of this system may attach directly to the discharge end of the barrel of syringe (1), for example, in embodiments where the electroseparation syringe does not comprise a needle.

Also shown in FIG. 2a, power source (11) is configured to connect with the first and second power supply connectors, which are provided by the wires extending from electroseparation syringe (1) to the terminals of power source (11).

In some embodiments, the system further comprises an analyser, such as an analyser as described above.

Also provided herein is an apparatus for analysing a sample comprising:
an electroseparation syringe comprising a syringe barrel and a plunger and a pair of electrodes positioned to enable a voltage to be applied across any liquid contained within the syringe barrel, or a receiver for receiving a separate electroseparation syringe;
a power supply for supplying a voltage potential;
a plunger controller (e.g. a pump) for operation of the plunger to draw up and dispense liquid into the syringe barrel;
an analyser for analysing liquid delivered to the analyser;
a sample reservoir for holding aqueous solution to be subjected to analysis;
a valve in fluid connection with the electroseparation syringe, that enables fluid flow between the electroseparation syringe and the analyser, and fluid flow between the electroseparation syringe and the sample reservoir; and
a controller for controlling operation of the power supply to the electrodes, operation of the plunger to draw liquids into the syringe barrel and dispense liquids from the syringe barrel, and to control the valve setting for controlling the direction of fluid flow.

The valve is suitably operated in the apparatus to control opening and closing of an inlet/outlet end of the syringe, in a sequence that enables a pressure to be applied (i.e. higher than the pressure applied during discharge of the fluid in the syringe through an open end of the syringe) during application of a voltage potential across liquid in the electroseparation syringe. The controller suitably controls the co-ordinated closing of the syringe inlet/outlet and operation (e.g. depressing, advancement or activation) of the plunger to apply pressure across liquid in the syringe during the application of a voltage potential across liquid in the syringe. As noted above, it has surprisingly been found that when the process is carried out under increased pressure improved analytical results are achieved. Without wishing to be bound by theory, the improved results are believed to be achieved by the critical suppression of bubble formation inside the syringe barrel during the electroseparation step due to the enhanced solubility of the generated gases under increased pressure. The apparatus with these equipment features, including the valve and control system to control operation of the valve and plunger to apply pressure during the application of the voltage potential, allows the performance of this process with the required increased pressure application. Without wishing to be bound by theory, the improved results are believed to be achieved by the critical suppression of bubble formation inside the syringe barrel during the electroseparation step due to the enhanced solubility of the generated gases under increased pressure.

As indicated above, the apparatus may come with an integrated electroseparation syringe, or the apparatus may comprise a receiver into which a separately-supplied electroseparation syringe is inserted, to allow connection of the electroseparation syringe to the power supply and other components of the apparatus. Even when supplied with the apparatus, the electroseparation syringe may be removable. Any suitable clips, connectors or fittings suitable for receiving the electroseparation syringe may be used.

The power supply allows the supply of a voltage potential across liquid contained within the syringe barrel in use, and therefore includes connectors to the electrodes of the electroseparation syringe. In embodiments where the apparatus contains a receiver for the electroseparation syringe, for the syringe to be inserted in place prior to operation, the power supply includes connectors associated with the receiver so that there is electrical connection between the power supply and the electrodes of the electroseparation syringe when the electroseparation syringe is received in the receiver.

The apparatus contains at least one reservoir, but may comprise a plurality of reservoirs, including the sample reservoir as one of the reservoirs. Other reservoirs may be for background electrolyte, waste, and washing solution. The washing solution is for washing the syringe between separate analyses. There may be multiple sample reservoirs. In this context, the term “reservoir” is used broadly to refer to any container or opening that allows for connection to a fluid receptacle. Thus, the reservoir may comprise a fluid tube or capillary that is inserted into a sample tube (e.g. a test tube) containing sample to be tested.

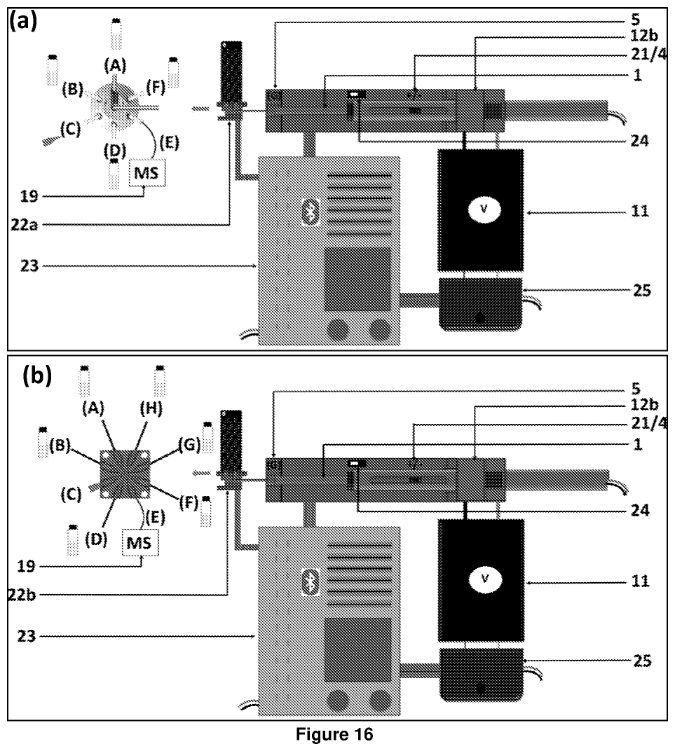

Two systems of this type are illustrated in FIGS. 16a and 16b. In these figures, the following features are shown:
Power Supply (11), including an input voltage module (25).
Syringe (1), which includes a syringe barrel (6) and electrodes (5, 21/4)—one (5) at the outlet (or inlet/outlet end) of the syringe barrel, and the other (21/4) associated with the plunger.
Pump (12b) with built-in points (connections) for high voltage application.
A valve, being either a 6-port valve (22a) or an 8-port valve (22b). In each figure, there is a second cross-sectional view of the valve showing each of the valve settings. In each case, there are valve settings marked A-F or A-H which are associated with different fluid reservoirs, plugs or analyser connections, including: background electrolyte reservoir (A), sample reservoir (B), a plug (C), waste reservoir (D), fluid connection to the analyser (E)—in this case, ESi-MS analyser (19); washing solution (F), and optional additional washing solutions (G) and (H).
Controller (23), in the form of a computer (with mother board), which controls the operation of the power supply, pump and valve. The controller may also operate or display the results of analysis performed by the analyser, and may be integrated with the analyser or separate from it.
A analyzer—in this case a mass spectrometer (MS) (19).

Any form of fluid passageways or capillary tubes may connect the various components of the system. There is also a button (24) marked on the syringe of the embodiments illustrated that serves to reverse the polarity of the voltage applied. In alternative arrangements, this is not present.

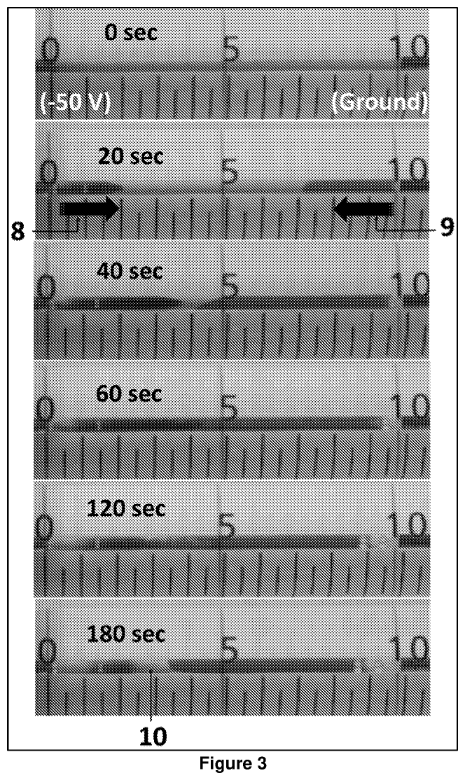

In the embodiments as illustrated in FIG. 16a, the following dimensions apply: The inlet/outlet of the syringe includes a needle with a length of 35 mm, internal diameter of 0.2 mm. The maximum capacity or volume of the syringe is 1.10 μL. The valve volume is 0.415 μL for the 6-port valve, and 0.37 μL for the 8-port valve. The sample reservoir fluid passageway has a length of 10 cm, internal diameter of 150 μm and holds a volume of 1.77 μL. The fluid passageway to the analyser at valve position 5 has a length of 25 cm, internal diameter of 50 μm and holds a volume of 0.49 μL. The total dead volume to MS is 2.01 μL in FIG. 16a and 1.96 μL in FIG. 16b, and the total dead volume to the BGE vial or the sample vial is 3.29 μL in FIG. 16a and 3.24 μL in FIG. 16b.

In the embodiment illustrated, and also generally with reference to other embodiments of the invention not illustrated, the controller may control the operation of a sequence of steps to wash the syringe between sample analyses.

To this end, the controller can be operated to perform a step of washing the syringe between sample analyses through controlled opening and closing of the syringe to the washing solutions and operation of the pump to draw up and expel the washing solutions to waste. The controller then operates to control the operation of the pump and valve to draw sample solution into the syringe barrel. Additional solutions (e.g. background electrolyte) solution may also be drawn up into the syringe in the arrangement required for performing the desired separation (e.g. an amount of sample solution may be sandwiched between amounts of BGE solution). The controller then operates to apply a voltage potential across the electrodes to effect a change in the distribution of the analyte being analysed in the sample. The controller then operates the valves and pump, and optionally also the power supply, to control the dispensing of the sample solution to the analyser via valve position E. There may be transmission of only a portion of the contents of the syringe barrel to the analyser, with other portions directed to waste at valve position D. The valve may also be operated to control the size of the opening through which the fluid needs to pass as it is dispensed from the syringe, to increase the pressure on the sample being dispensed form the syringe barrel and to the analyser.

### EXAMPLES

The invention will be further described by way of non-limiting example(s). It will be understood to persons skilled in the art of the invention that many modifications may be made without departing from the spirit and scope of the invention.

### Example 1—NRB Formation

This experiment confirms the formation of a NRB within an electroseparation syringe. This experiment also confirms the mechanism of NRB formation.

A syringe was filled with 10.0 μL of a solution containing 80% (v/v) universal indicator and 5.0 mM NH₄Ac, pH 7, after coating the syringe barrel dynamically with 1.0% (w/v) hydroxypropyl methylcellulose (HPMC). The application of −50 V resulted in the generation of a H⁺ flux from the ground (red colour) and OH⁻ ions flux from the cathode (purple colour). A stable NRB was formed within 3 minutes using the mentioned conditions. Results of this experiment are shown in FIG. 3.

### Example 2—Focussing of Proteins Using the Electroseparation Syringe

A similar procedure as described in Example 1 was employed to show the focussing of Chromeo™ labelled bovine serum albumin (pl 4.7) into a NRB using applied voltage of −50 V, 5.0 mM NH₄Ac (pH8) as a background electrolyte and an HPMC (1.0% (w/v)) coated electroseparation syringe.

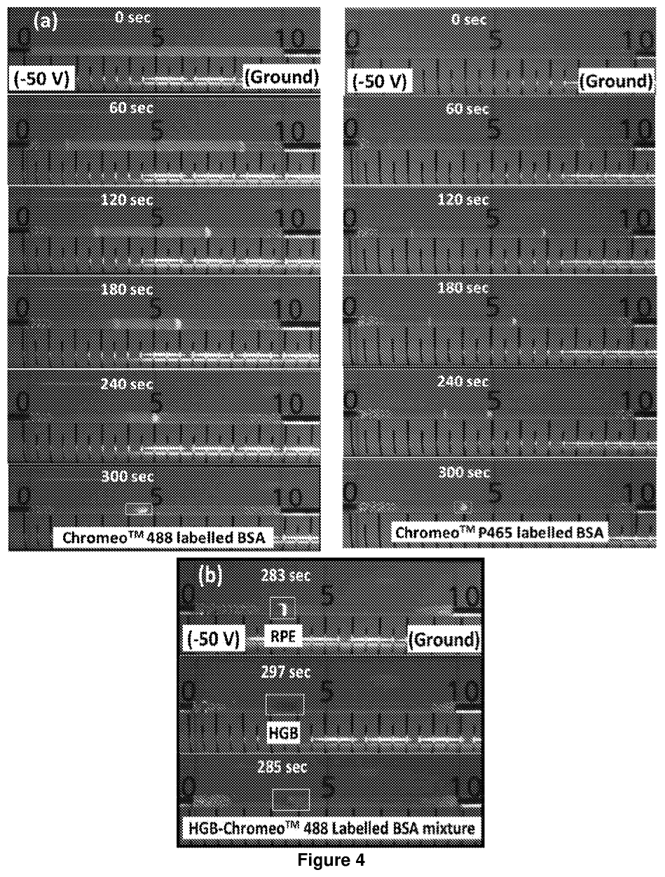

FIG. 4a shows the results of this experiments where at the start of the experiment (time (min:sec)=0:00) BSA is evenly distributed throughout the aqueous solution contained in the syringe as the fluorescence is substantially constant throughout the solution. A voltage of −50V is applied to the needle of the syringe (left hand side). As the voltage is applied, the fluorescing region of the solution is seen to focus inwards from both ends to a single band between the 4-5 μL marks on the syringe within five minutes.

Similar results have been obtained to focus different proteins with different pl values where FIG. 4b indicates focusing of R-phycoerythrin (RPE, pl 4.2, 40.0 μg/mL), haemoglobin (HGB, μl 6.9, 350.0 μg/mL), and a mixture of Chromeo™ 488 labelled BSA (100 μg/mL) and HGB (350.0 μg/mL).

### Example 3—Analytical Process for Detection of Histidine Concentration Using an Electroseparation Syringe

The electroseparation syringe (ES) was coupled with an ESI-MS system for histidine determination in standard solutions and in spiked urine samples. The experimental conditions for the determination of histidine with the ES-ESI-MS system are summarized in Table 1 (below).

TABLE 1
Optimum experimental conditions for the determination of
histidine with electroseparation syringe- ESI-MS system

| | 1.0 mM ammonium acetate |
|---|---|
| Background electrolyte | in 70.0 % (v/v) Acetonitrile |
| Starting pH | 4.0 |
| IEF syringe flow rate | 4.0 |
| (infusion step) (μL/min) | |
| Applied voltage: IEF | −200.0/−400.0 |
| step/Infusion step (V) | |
| Time for the IEF step (mins) | 5.0 |
| Sheath liquid composition | 0.2% (v/v) formic acid |
| Sheath liquid flow rate | 4.0 |
| (μL/min) | |
| Nebulizer gas pressure | 5.0 |
| (psi) | |
| Dry gas flow rate (L/min) | 4.0 |
| Dry gas temperature (° C.) | 365.0 |

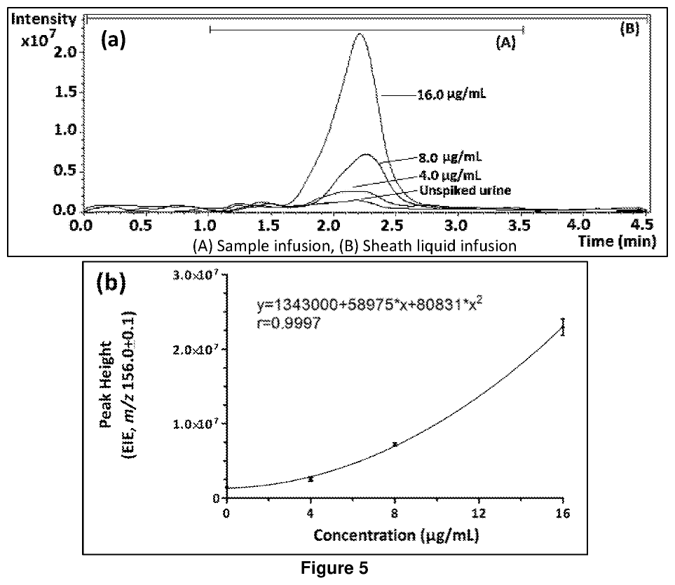

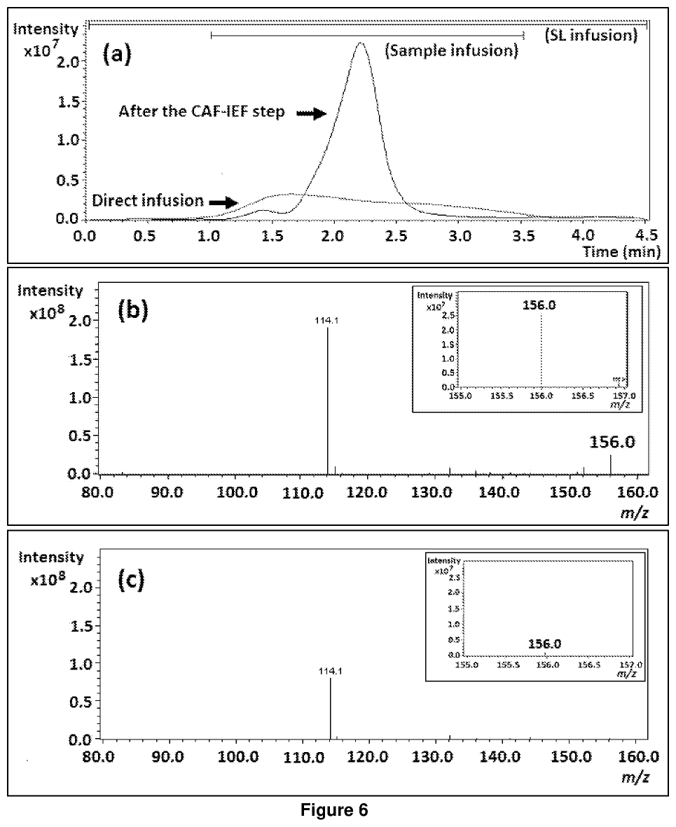

The results of this process are shown in FIGS. 5a, 5b and 6a-c. The analyser used in this example is an ESI-MS detector. The focused histidine elutes at around 2.25 minutes (FIG. 5a), and the intensity of detection correlates with the concentration of histidine spiked into the sample (FIG. 5b). For histidine determination in standard solutions, the plot of peak height (EIEs, m/z 156.0±0.1) versus calibrator concentration was fit with a non-linear quadratic equation (y=−1922000+1450000*x+51918*x²) with a range of 4.0-64.0 μg/mL and correlation coefficient(r)=0.9998. The method accuracy and precision data were determined at 4.0, 8.0, 16.0, 32.0, and 64.0 μg/mL using three replicates to demonstrate an accuracy of 91.86% to 102.16% and a precision with % Error less than 6% as shown in Table 2 (below). The developed ES approach allowed a very simplified protocol for histidine determination in spiked urine samples where the ES was used to dilute the urine sample 10 times by the background electrolyte (BGE), histidine focusing and infusion to the ESI-MS. A quadratic fit calibration curve was constructed (y=1343000+58975*x+80831*x²) (r=0.9997) for histidine determination in urine samples (FIG. 5b) with accuracy ranged from 88.25% to 102.16%. The precision data was found to be satisfactory with relative standard deviation (RSD) less than 11% and % Error less than 7% (Table 2).

This example also shows that a concentration factor of 8.5 folds was achieved for the determination of histidine in spiked urine samples using the developed electroseparation syringe—ESI-MS approach as illustrated in FIG. 6. FIG. 6a shows the extracted ion electropherograms (EIEs) (m/z 156.0±0.1) of spiked urine sample with a final added concentration of 16.0 μg/mL histidine where (A) after subjection to the focussing step of the process of the invention, and (B) without the focussing step. FIG. 6b shows the mass spectrum obtained from the peak at time=2.25 from the EIE shown in FIG. 6a (A) and FIG. 6c shows the mass spectrum obtained from the peak at time=2.25 from the EIE shown in FIG. 6a (B). The enhanced sensitivity illustrates the significance of the process of the invention to preconcentrate the target analyte and to reduce matrix suppression.

TABLE 2
Accuracy and precision for the determination of histidine with the IEF syringe-ESI-MS
approach*

| | | | Intra-day precision | | Inter-day precision | |
|---|---|---|---|---|---|---|
| | Concentration | % | RSD (%)** | | RSD (%) | |
| Matrix | (μg/mL) | Recovery | (n = 3) | % Error*** | (n = 3) | % Error |
| Standard | 4.0 | 94.72 | 3.54 | 2.04 | 7.58 | 4.38 |
| solutions | 8.0 | 91.86 | 8.37 | 4.83 | 4.72 | 2.47 |
| | 16.0 | 92.98 | 7.14 | 4.13 | 8.74 | 5.05 |
| | 32.0 | 102.16 | 4.33 | 2.50 | 6.10 | 3.52 |
| | 64.0 | 99.89 | 0.69 | 0.40 | 9.90 | 5.72 |
| Spiked | Concentration | | Intra-day precision | | Inter-day precision | |
| urine | added | | RSD (%) | | RSD (%) | |
| samples | (μg/ml) | % Found | (n = 3) | % Error | (n = 3) | % Error |
| | 4.0 | 88.25 | 9.86 | 5.69 | 10.66 | 6.16 |
| | 8.0 | 102.16 | 3.14 | 1.81 | 9.97 | 5.76 |
| | 16.0 | 99.91 | 4.82 | 2.78 | 9.24 | 5.34 |

*The % found and relative standard deviation values met the requirements of bioanalytical guidance [F.D.A. U.S. Department of Health and Human Services, Center For Drug Evaluation and Research Center of Veterinary Medicine, Guidance for Industry: Bioanalytical Method Validation [Draft Guidance] (2013). https://www.fda.gov/downloads/drugs/guidances/ucm368107.pdf]
**relative standard deviation, RSD = 100 sample standard deviation /mean
***% Error = (% RSD/√n)

### Example 4—Application of the Electroseparation Syringe in the Detection of Charged Analytes (Clean-Up of Biological Samples Using the Electroseparation Syringe)

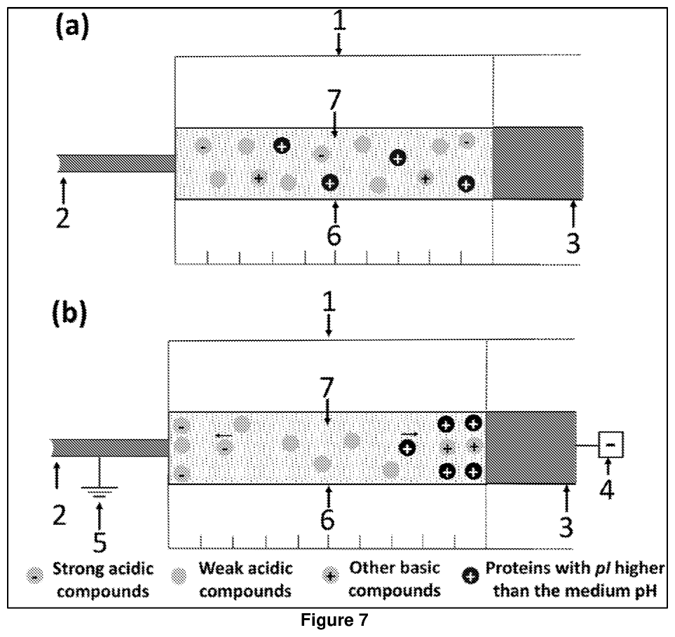

The clean-up of serum samples from interfering proteins is based on the difference in the ionisation between the serum proteins and the target analytes upon dilution in an aqueous solution having a certain pH value using the ES. In this example, the aqueous solution comprises 50 mM formic acid and has a pH of 2.5. This pH is lower than the pls of almost all the serum proteins ensuring that the serum proteins are positively charged, also ensuring all acidic compounds are uncharged, partially negatively charged or fully negatively charged based on their pKa values. Upon voltage application, all the positively charged serum proteins are focused and aggregated close to the syringe plunger while weakly acidic compounds are unfocused or partially focused toward the needle. This process is shown in schematic in FIG. 7.

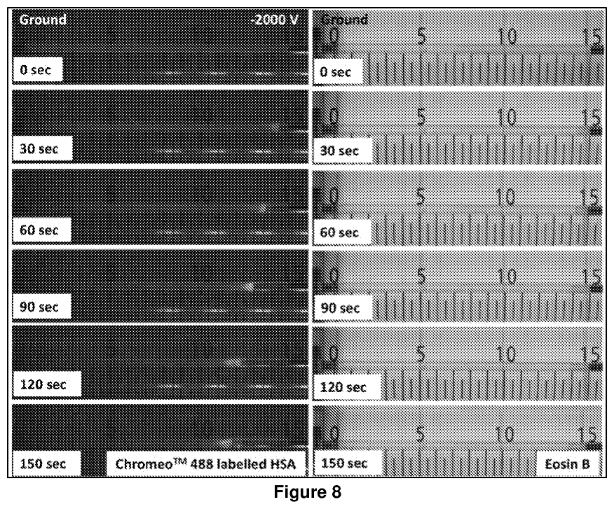

FIG. 8 shows the focusing of 200.0 μg/mL of Chromeo™ 488 labelled human serum albumin (HSA) (pl 4.7) to the plunger as a cathode versus the constancy of a weak acidic dye (eosin B, 100.0 μg/mL, pKa values=2.2 and 3.7) using an aqueous solution comprising 50 mM formic acid (pH 2.5) in 30% (v/v) acetonitrile.

This Example shows the ability to separate two different molecules into different regions of the aqueous solution using the processes of the invention. By focussing the concentration of at least one analyte or interfering molecules into a separate zone, the molecules are separated.

### Example 5—Analytical Process for the Detection of Neutral Analytes (Naproxen (NAP) and Paracetamol (PCM)) in the Presence of an Amphiphilic Molecule (HSA) in a Spiked Biological Sample

This Example will show that the processes of the invention can be used to detect various analytes with minimal matrix effect.

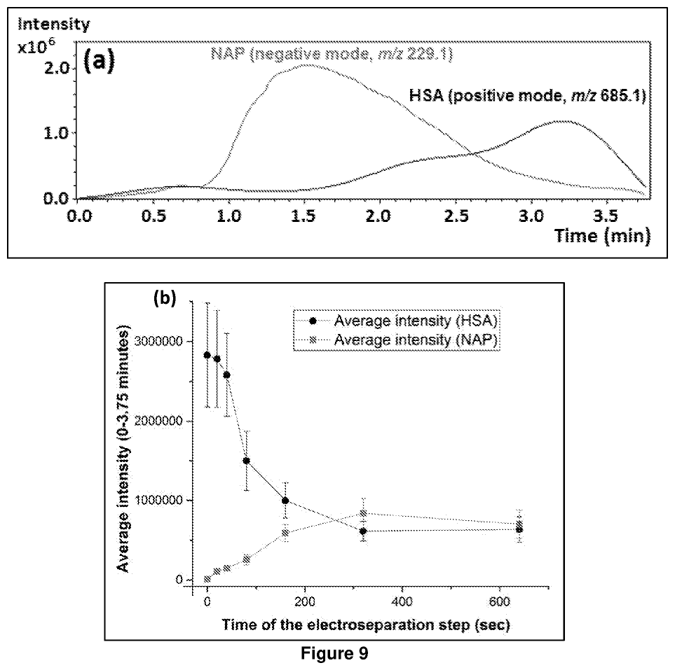

The concept of serum protein elimination from a sample containing a weakly acidic target compound using the invention was verified by ESI-MS analysis of a NAP/HSA mixture without the electroseparation step and after application of the electroseparation step for different time intervals. For NAP, EIEs were obtained for the molecular ion [M−1]-1 (m/z 229.1) and for HSA analysis, quantitation was achieved using source-induced fragmentation by adjusting voltage difference between the capillary exit and the skimmer to be 335 V. The resulting b₂₄⁴⁺ fragment ion (most abundant fragment, m/z685.1) was detected utilizing a positive mode of ionization. FIG. 9a depicts the focusing and aggregation of HSA toward the plunger (EIE: positive mode, m/z 685.1) by maintained application of −2000 V for 10 minutes. The signal intensity of NAP (EIE: negative mode, m/z229.1) was increased after HSA removal from this portion of the solution. A summary of this experiment is illustrated in FIG. 9b after integrating the mass spectra and plotting the average signals intensities vs time. The NAP signal increases with the time of the electroseparation step until about 320 seconds indicating a gradual removal of proteins over this time period. Application of voltage between 320-640 seconds did not result in a significant change in NAP signal. Accordingly, 320 seconds was selected as the time used for the electroseparation step in this example. Also, FIG. 9b shows the reverse proportional relationship between the average intensities of NAP and HSA signals due to the ionization suppression caused by HSA.

Protocol for serum spiking and analysis using the electroseparation syringe coupled with ESI-MS:
1—Collection of blood sample by pinprick a fingertip of a healthy adult volunteer.
2—Allow the blood to clot by leaving it undisturbed at room temperature. for 30 minutes.
3—Remove the clot by centrifugation at 6400 RPM for 10 minutes.
4—Transfer the liquid component (serum) into a clean Eppendorf tube.
5—Spiked serum samples were prepared by adding specific volumes of the standard solutions of NAP and PCM to the serum samples to obtain final concentrations of 4.0, 8.0, 16.0, 32.0, and 64.0 μg/mL of NAP, and 3.0, 6.0, 12.0, 24.0, and 48.0 μg/mL of PCM, respectively. Each serum sample was also spiked with 160.0 μg/mL of valproic acid as an internal standard (IS) and finally vortex-mixed for 30 seconds.
6—Background electrolyte (BGE) comprises 53.3 mM formic acid in 32.0% (v/v) ACN to give a final BGE composition of 50.0 mM formic acid in 30.0% (v/v) acetonitrile (after 15-fold dilution of the spiked serum with BGE)
7—15-fold dilution of the spiked serum with the BGE by aspirating of 1.0 μL of the spiked serum between 4.0 and 11.0 μL of the BGE.
8—Clean up step (electroseparation of proteins): −2000 V for 320 sec. 9—Infusion to ESI-MS with a flow rate 4 μL/min with a sheath liquid: ISP 80% (v/v), flow rate 10 μL/min.

The developed ES-ESI-MS approach offered a simplified protocol for the simultaneous determination of NAP and PCM in spiked serum samples using valproic acid as IS.

The parameters used for the clean-up step and ESI-MS analysis of the spiked serum samples are summarised in Table 3.

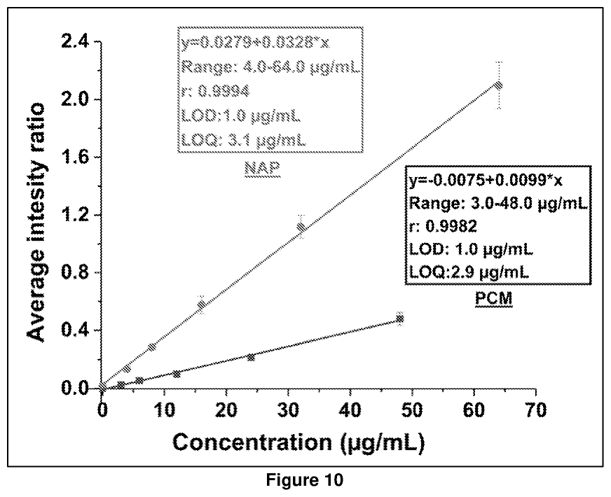

A linear correlation was achieved by plotting the average intensity ratio (each drug/IS) versus the added drug concentration as shown in FIG. 10. The corresponding regression equations were y=0.0279+0.0328*x for NAP and y=−0.0075+0.0099*x for PCM, with a regression correlation coefficient (r)=0.9994 for NAP and (r)=0.9982 for PCM.

The constructed calibration curves were developed based on 5 concentration levels to give concentration ranges of 4.0-64.0 μg/mL for NAP and 3.0-48.0 μg/mL for PCM, in addition to, unspiked serum sample. The lower level of quantitation (LLOQ) values for NAP and PCM were selected to give responses more than 5 times the response of the serum blank following FDA guidance provided in the U.S. Department of Health and Human Services, Center for Drug Evaluation and Research, Center for Veterinary Medicine, Guidance for Industry, Bioanalytical Method Validation [Draft Guidance], September 2013.

The accuracy of the developed method for the simultaneous determination of NAP and PCM in the spiked serum samples was evaluated at 5 different concentration levels; 4.0, 8.0, 16.0, 32.0, and 64.0 μg/mL for NAP and 3.0, 6.0, 12.0, 24.0 and 48.0 μg/mL for PCM (n=3) by calculating the ratio of the found and the added concentrations. The precision of the method (intra-day and inter-day) was expressed by the relative standard deviation (RSD) and % Error using the standard deviation and the mean of the assayed triplicates of each concentration. The method accuracy was from 81.23% to 104.79% for NAP and from 90.98% to 115.52% for PCM and the method precision had a % Error less than 11% and 10% for NAP and PCM, respectively as shown in Table 4.

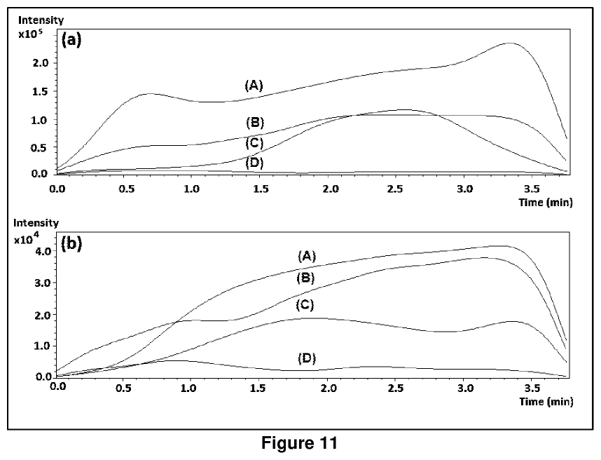

The process efficiency and the ion suppression were evaluated using four sample sets; set A included neat standard solutions, 15-fold dilution in the aqueous solution with the ES and infused to the ESI-MS, set B included neat standard solutions like set A but after the application of electroseparation step, set C included pre-spiked serum samples after the electroseparation step application, and set D included pre-spiked serum samples diluted in the aqueous solution and injected without the electroseparation step. Every set was made in triplicate for three different concentration levels within the determination ranges of NAP and PCM; 4.0, 16.0, 64.0 for NAP and 3.0, 12.0, and 48.0 μg/mL for PCM, respectively. The EIEs representing the four sets are summarized in FIG. 11 where FIG. 11a represents the EIEs (m/z 229.1, [M−1]⁻) of 16.0 μg/mL NAP and FIG. 11b represents the EIEs (m/z 150.2, [M−1]⁻) of 12.0 μg/mL PCM in the four sets (A-D)

Process efficiency (% PE) was assessed by comparing the average intensity for the whole run between set C and set A: PE (%)=C/Ax100.

Ion suppression due to the serum matrix (a metric indicating reduction of the matrix effect) was evaluated as follows: Ion suppression=(100−(D/Ax100)). The process efficiency of the clean-up step and the ion suppression due to the serum matrix were evaluated at three concentration levels of each drug as summarized in Table 5. The average process efficiency for NAP was found to be 39.91% giving a signal 6.8 folds higher than the infusion of spiked serum without the clean-up step (average ion suppression without the clean-up step=94.09%). Likewise, the average process efficiency for PCM was 36.09% and the PCM signal was 5.9 folds higher after the clean-up step compared to infusion without the clean-up step where the ion suppression was 93.91%.

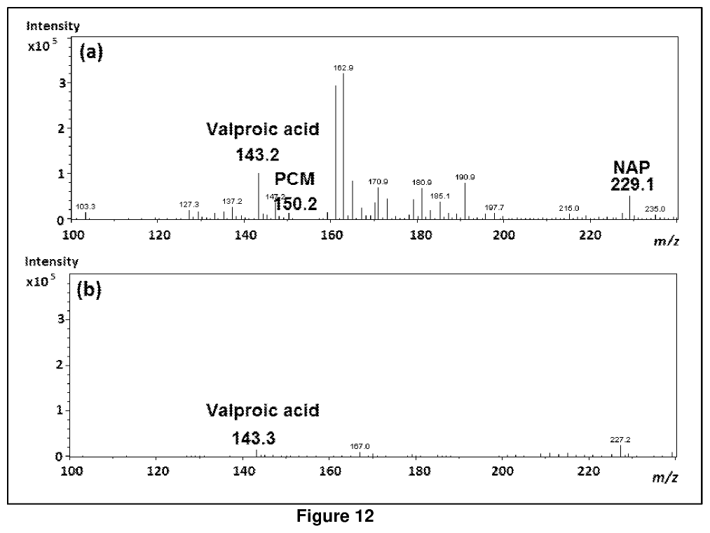

The impact of the clean-up step on mass spectrum sensitivity can be seen by comparing the mass spectra shown in FIG. 12a and FIG. 12b, where FIG. 12a shows a mass spectrum obtained following the clean-up step and FIG. 12b shows the results without the clean-up step on the sample.

TABLE 3
Parameters for the analysis of spiked serum samples

| Composition of the | 50.0 mM formic acid |
|---|---|
| aqueous solution | in 30% (v/v) acetonitrile. |
| Electroseparation syringe | 4.0 |
| flow rate (μL/min ) | |
| Sheath liquid composition | Isopropanol, 80% (v/v) |
| Sheath liquid | 10.0 |
| flow rate (μL/min ) | |
| Nebulizer gas pressure (psi) | 7.5 |
| Dry gas flow rate (L/min) | 5.0 |
| Dry gas temperature (° C.) | 225.0 |
| Applied voltage (V) | −2000/−500 |
| Electroseparation step/ | |
| Infusion step | |
| Time of the electroseparation | 320 |
| step (sec) | |

TABLE 4
Accuracy and precision for the determination of naproxen and paracetamol in spiked
serum samples using an electroseparation syringe coupled with an ESI-MS analyser (ES-ESI-
MS)

| | Concentration | | Intra-day precision | | Inter-day precision | |
|---|---|---|---|---|---|---|
| | spiked | | *RSD (%) | | *RSD (%) | |
| Drug | (μg/mL) | % Found | (n = 3) | **% Error | (n = 3) | **% Error |
| NAP | 4.0 | 81.23 | 6.10 | 3.52 | 18.91 | 10.92 |
| | 8.0 | 97.98 | 7.06 | 4.08 | 8.69 | 5.02 |
| | 16.0 | 104.79 | 10.52 | 6.07 | 14.34 | 8.28 |
| | 32.0 | 104.30 | 7.22 | 4.17 | 13.08 | 7.55 |
| | 64.0 | 98.91 | 7.63 | 4.41 | 10.48 | 6.05 |
| PCM | 3.0 | 115.52 | 11.17 | 6.45 | 17.28 | 9.98 |
| | 6.0 | 104.98 | 11.58 | 6.69 | 16.70 | 9.64 |
| | 12.0 | 90.98 | 10.14 | 5.86 | 14.86 | 8.58 |
| | 24.0 | 93.96 | 9.47 | 5.47 | 10.88 | 6.28 |
| | 48.0 | 102.51 | 9.15 | 5.28 | 10.62 | 6.13 |

Notes:
The accuracy and precision are not beyond the limits of the FDA guidance for the bioanalytical method validation.
*relative standard deviation, RSD = 100 S/x’
**% Error = (% RSD/√n)

TABLE 5
Process efficiency (PE) data for NAP and PCM in spiked serum samples.

| | | | | | | | ***Ion |
|---|---|---|---|---|---|---|---|
| | | | | | | **Process | suppression |
| | | | | | | efficiency | without the |
| | Concentration | Average signal intensity, (n = 3) | | | | (% PE) | clean-up step |
| Drug | (μg/mL) | Set A | *Set B | Set C | Set D | % | % |
| NAP | 4.0 | 11233 | 8581 | 4094 | 905 | 36.45 | 91.94 |
| | 16.0 | 127272 | 94863 | 50786 | 6562 | 39.90 | 94.84 |
| | 64.0 | 354204 | 242285 | 153615 | 15992 | 43.37 | 95.49 |
| | | | | | | Mean ± SD | Mean ± SD |
| | | | | | | 39.91 ± 3.46 | 94.09 ± 1.89 |
| PCM | 3.0 | 3353 | 2116 | 1229 | 240 | 36.65 | 92.84 |
| | 12.0 | 33004 | 26000 | 13168 | 1220 | 39.90 | 96.30 |
| | 48.0 | 55828 | 35541 | 17711 | 4140 | 31.72 | 92.58 |
| | | | | | | Mean ± SD | Mean ± SD |
| | | | | | | 36.09 + 4.12 | 93.91 + 2.08 |

Notes:
*Set B includes the analyte standards with the clean-up step.
**Process efficiency = C/A × 100
Where C is the mean average signal intensity of the pre-spiked serum samples with the clean-up step (set C) and A is the mean average signal intensity of the same analyte standards (set A).
***Ion suppression = (100 − (D/A × 100))

### Example 6—Analytical Process for the Detection of Neutral Analytes (Clomipramine, Chlorphenamine, Pindolol and Atenolol) in the Presence of an Amphiphilic Molecules (Serum Proteins) in a Spiked Serum Sample by Tandem Mass Spectrometry

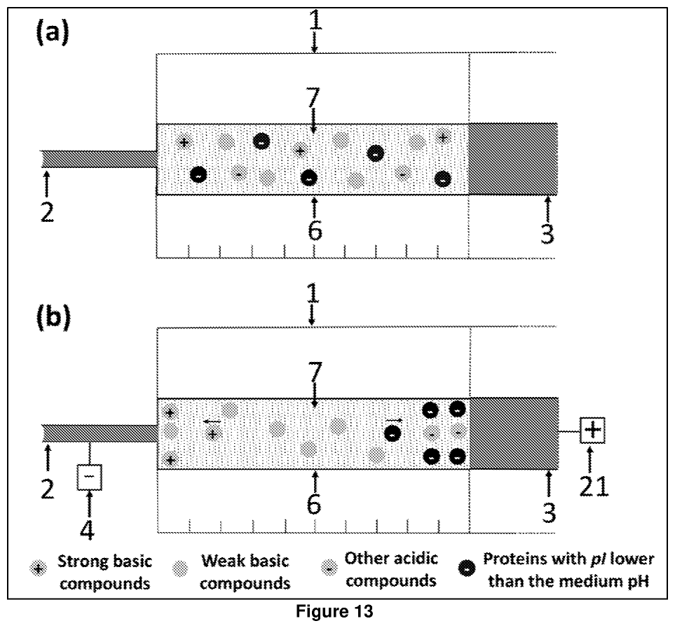

In this example, the aqueous solution comprises 300 mM ammonium hydroxide and 30% (v/v) acetonitrile and has a pH of 11.4. This pH is higher than the pls of almost all the serum proteins ensuring that the serum proteins are negatively charged, also ensuring all basic compounds are uncharged, partially positively charged or fully positively charged based on their pKa values. Upon voltage application using the needle as a cathode and the plunger as an anode, all the negatively charged serum proteins are focused and aggregated close to the syringe plunger while weakly basic compounds (clomipramine, chlorphenamine, pindolol and atenolol) are unfocused. This process is shown in schematic in FIG. 13.

The MS/MS scans were done in the positive mode using a fragmentation time of 50 ms, an isolation width of 4 mass units, and fragmentation amplitude of 0.5 V for clomipramine, chlorphenamine, and atenolol and 0.4 V for pindolol. With multiple reaction monitoring (MRM), m/z of 315>86, m/z 275>230, m/z 249>116, and m/z 267>190 were used for the detection of clomipramine, chlorphenamine, pindolol and atenolol, respectively.

Spiked serum samples were prepared by adding specific volumes of the standard solutions to obtain spiked concentrations of 80.0, 10.0, 50.0, 250.0 ng/mL of clomipramine, chlorphenamine, pindolol and atenolol, respectively (spiking has been done at levels lower than the maximum plasma concentrations of all drugs). Each serum sample was diluted five times in the BGE. 10 μL of the diluted serum was aspirated by the electroseparation syringe and 800 V was applied for 90 seconds using the plunger as an anode and the needle as a cathode to clean up the serum samples from the serum proteins followed by Infusion to ESI-MS with a flow rate of 5 μL/min while keeping 200 V applied. A sheath liquid of 0.5% (v/v) formic acid in 75% (v/v) methanol was being infused coaxially to the sprayer, flow rate 5 μL/min.

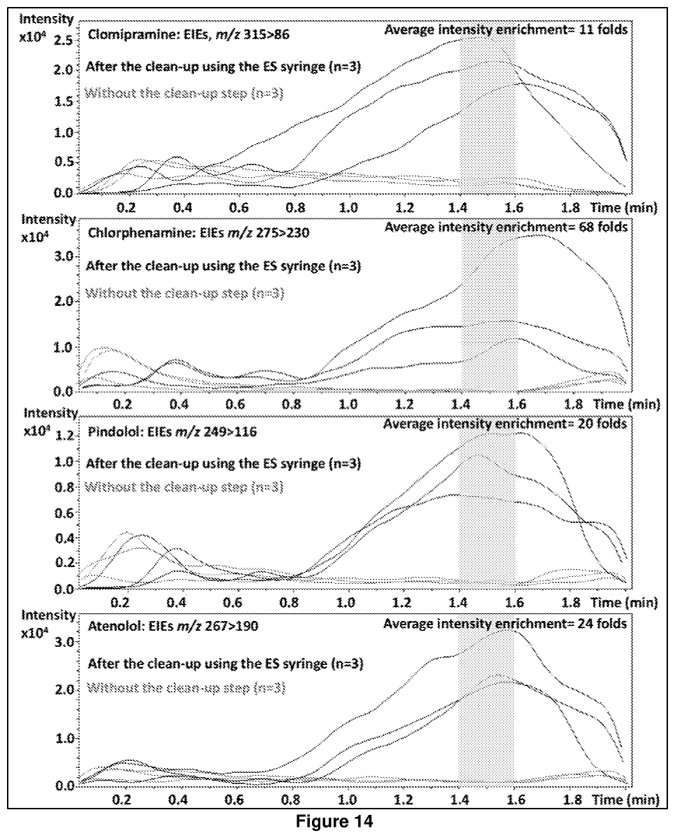

The significance of the clean-up step using the electroseparation syringe has been shown in FIG. 14 by comparing the EIEs of each drug after the electroseparation step and without the electroseparation step (n=3). Using the time interval of 1.4 to 1.6, the signal intensity was enhanced by 11 folds for clomipramine, 68 folds for chlorphenamine, 20 folds for pindolol, and 24 folds for atenolol.

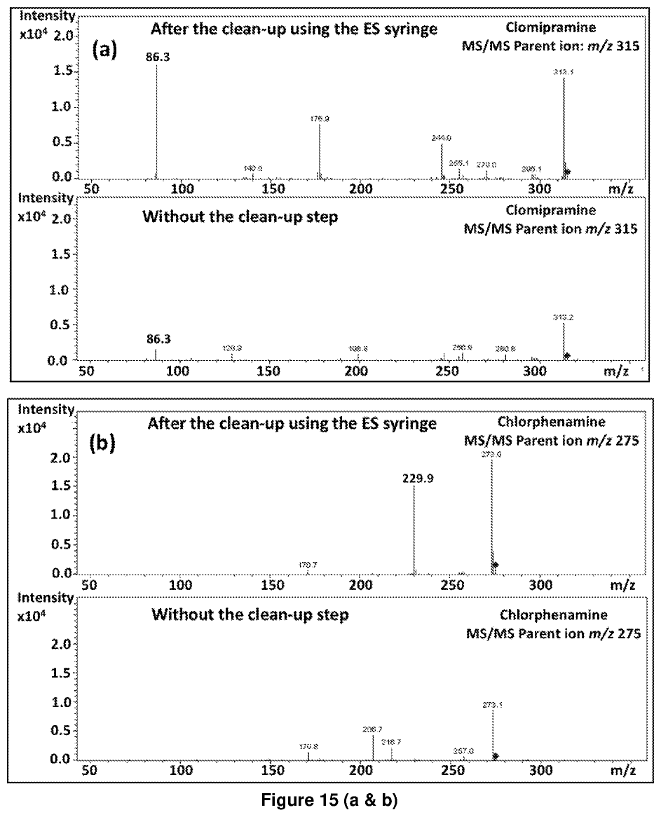

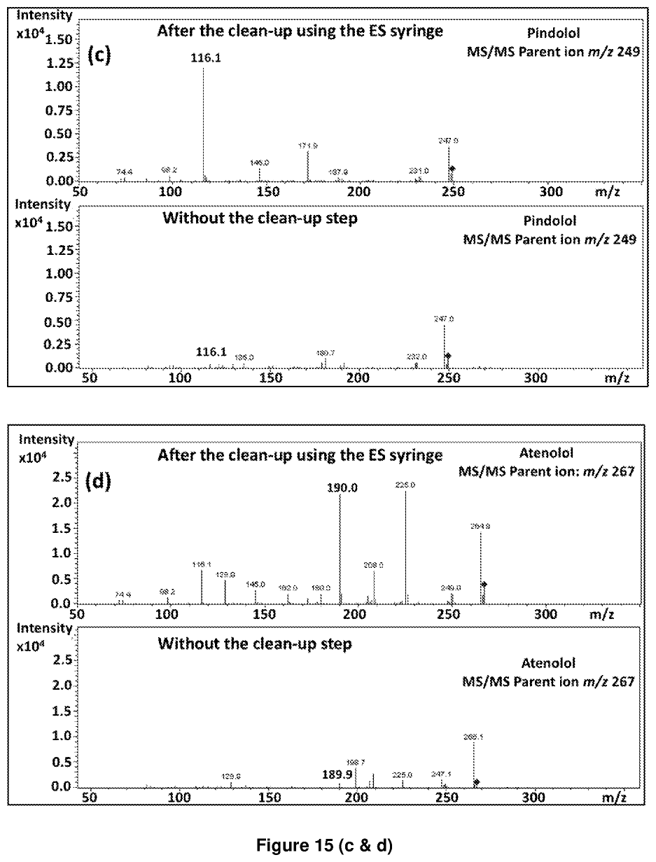

FIGS. 15a-d indicate the MS/MS spectra of clomipramine, chlorphenamine, pindolol, and atenolol, respectively, after the clean-up step using the electroseparation syringe and without the clean-up step.

### Items

1. A process for modifying the distribution of a compound in a solution comprising:
drawing the solution comprising the compound into an electroseparation syringe comprising a syringe barrel, a plunger and electrodes positioned to apply a voltage across solution contained in the syringe barrel, and
applying a voltage across the solution in the syringe barrel to modify the distribution of the compound within the solution contained in the syringe barrel.
   2. The process of item 1, wherein the compound is an analyte.
   3. The process of item 2, wherein the modifying of the distribution of the analyte within the solution contained in the syringe barrel comprises:
focusing a concentration of the analyte within a region of the solution contained in the syringe barrel, or
generating a region within the solution with an increased concentration of the analyte in the solution, wherein the analyte is a net neutral molecule; or
separating the analyte from net neutral compounds also contained within the solution, wherein the analyte is a charged compound.
   3a. The process of claim 1, wherein the solution comprises the compound and an analyte, and the modifying of the distribution of the compound within the solution contained in the syringe barrel comprises focusing an increased concentration of the compound within one region of the solution to create another region of solution containing the analyte with a depleted concentration of the compound.
   4. The process of any one of items 2 to 3a, comprising:
injecting the solution into an analyser to allow analysis of the analyte.
   5. The process of any one of items 2 to 4, wherein the voltage applied to the solution is a voltage within the range of −1.23V to about −5000 V, or within the range of +1.23V to about 5000V.
   6. The process of any one of items 2 to 5, wherein a second voltage is applied during the step of injecting the solution into the analyser.
   7. The process of item 6, wherein the second voltage is a voltage in a range of about −1.23V to about −1000V or within the range of about +1.23V to about +1000V.
   8. The process of any one of items 2 to 7, wherein prior to applying the voltage across the aqueous solution, the pressure within a barrel of the electroseparation syringe is increased.
   9. The process of item 8, wherein the electroseparation syringe comprises a valve associated with a discharge end of the barrel, and the pressure is increased by applying pressure on the plunger of the electroseparation syringe when the valve is closed.
   10. The process according to any one of items 2 to 9, wherein:
the analyte is an amphiphilic analyte, or
the analyte is a neutral analyte or a charged analyte, and the solution comprises an amphiphilic molecule.
   11. The process according to item 10, wherein the amphiphilic analyte or amphiphilic molecule is selected from a protein, a peptide and an amino acid.
   12. The process of any one of items 1 to 11, wherein the solution comprises a background electrolyte selected from the group consisting of ammonium salts, carboxylic acids, carboxylate salts, amines and combinations of one or more thereof.
   13. A process for determining a concentration of an analyte in an aqueous solution, comprising:
a. applying a voltage across the aqueous solution comprising the analyte and a background electrolyte in an electroseparation syringe, the electroseparation syringe comprising a syringe barrel, a plunger and a pair of electrodes positioned to apply the voltage across the aqueous solution in the syringe barrel; and
b. injecting the aqueous solution into an analyser.
   14. A process for focussing a concentration of a molecule in an aqueous solution, comprising:
a. applying a voltage across the aqueous solution comprising the molecule and a background electrolyte in an electroseparation syringe to generate a region comprising an increased concentration of the molecule in the solution, the electroseparation syringe comprising a syringe barrel, a plunger and a pair of electrodes positioned to apply the voltage across the aqueous solution.
   15. A process for separating a charged compound from an amphiphilic compound, the process comprising applying a voltage across an aqueous solution comprising the charged compound, the amphiphilic compound and a background electrolyte in an electroseparation syringe comprising a syringe barrel, a plunger and a pair of electrodes positioned to apply the voltage across the aqueous solution.
   16. An electroseparation syringe comprising a syringe barrel, a plunger, and a pair of electrodes, wherein the electrodes are configured to come into electrical contact with a solution contained within the syringe barrel in use, so as to enable a voltage to be applied longitudinally across solution contained in the syringe barrel.
   17. The electroseparation syringe of item 16, wherein the pair of electrodes comprise:
a cathode comprising a first power supply connector; and
an anode comprising a second power supply connector.
   18. The electroseparation syringe of item 17, wherein the syringe barrel has a discharge end and a plunger receiving end, and one of the cathode or the anode is positioned at the discharge end of the barrel.
   19. The electroseparation syringe of any one of items 16 to 18, wherein the plunger comprises one of the electrodes.
   20. The electroseparation syringe of any one of items 16 to 19, wherein one of the electrodes is in the form of a needle.
   21. The electroseparation syringe of any one of items 16 to 20, wherein the syringe barrel comprises an internal coating.
   22. The electroseparation syringe of any one of items 16 to 21, comprising a valve associated with the discharge end of the barrel.
   23. The electroseparation syringe of any one of items 16 to 22, with a capacity of between 0.5 μl and 1 ml, such as between 0.5 μl and 20p.
   24. An apparatus for analysing a sample comprising:
an electroseparation syringe comprising a syringe barrel, a plunger and a pair of electrodes positioned to enable a voltage to be applied across any liquid contained within the syringe barrel, or a receiver for receiving an electroseparation syringe;
a power supply for supplying a voltage potential;
a plunger controller for operation of the plunger to draw up and dispense liquid into the syringe barrel;
an analyser for analysing liquid delivered to the analyser;
a sample reservoir for holding solution to be subjected to analysis;
a valve in fluid connection with the electroseparation syringe, that enables fluid flow between the electroseparation syringe and the analyser, and fluid flow between the electroseparation syringe and the sample reservoir; and
a controller for controlling operation of the power supply to the electrodes, operation of the plunger to draw liquids into the syringe barrel and dispense liquids from the syringe barrel, and to control the valve setting for controlling the direction of fluid flow.
   25. The apparatus of item 24, wherein the electroseparation syringe is as defined in any one of items 16 to 21.
   26. The apparatus of item 24 or item 25, when used to perform the process of any one of items 1 to 15.
   27. An analytical system, comprising:
an electroseparation syringe comprising a syringe barrel, a plunger, and a pair of electrodes positioned to apply a voltage across an aqueous solution contained in the syringe barrel in use;
a power supply configured to connect with the electrodes; and
an analyser adapted to receive and analyse an analyte from the electroseparation syringe.
   28. The analytical system of item 27, wherein the electroseparation syringe is as defined in any one of items 16 to 21.
   29. The analytical system of item 27 or item 28, when used to perform the process of any one of items 2 to 15.
   30. The process, electroseparation syringe, apparatus or system as described above and substantially as herein described with reference to one or more of the Figures.
   31. A process for determining a concentration of an analyte in an aqueous solution, comprising:
a. applying a voltage across the aqueous solution comprising the analyte and a background electrolyte in an electroseparation syringe, the electroseparation syringe comprising an anode and a cathode positioned to apply the voltage across the aqueous solution; and
b. injecting the aqueous solution into an analyser.
   32. The process of item 31, wherein the analyte is an amphiphilic analyte.
   33. The process of item 31, wherein the analyte is a neutral analyte or a charged analyte.
   34. The process of item 31 or 33, wherein the solution further comprises an amphiphilic molecule.
   35. The process of item 32 or 34, wherein the amphiphilic analyte or amphiphilic molecule is selected from a protein, a peptide and an amino acid.
   36. The process of any one of items 31 to 35, wherein the background electrolyte comprises an ammonium salt, a carboxylic acid, a carboxylate salt and an amine or a combination thereof.
   37. The process of any one of items 31 to 36, wherein the voltage applied to the solution is from about −5000 V to about 5000V.
   38. The process of any one of items 31 to 37, wherein a second voltage is applied during the step of injecting the solution to the analyser.
   39. The process of item 38, wherein the second voltage is from about −1000V to about 1000V.
   40. The process of any one of items 31 to 39, wherein prior to applying the voltage across the aqueous solution, the pressure within a barrel of the electroseparation syringe is increased.
   41. The process of item 40, wherein the electroseparation syringe further comprises a valve at a discharge end of the barrel, and the pressure is increased by depressing a plunger of the electroseparation syringe when the valve is closed.
   42. A process for focussing a concentration of a molecule in an aqueous solution, comprising:
a. applying a voltage across the aqueous solution comprising the molecule and a background electrolyte in an electroseparation syringe to generate a region comprising an increased concentration of the molecule in the solution, the electroseparation syringe comprising an anode and a cathode positioned to apply the voltage across the aqueous solution.
   43. A process for separating a charged compound from an amphiphilic compound, the process comprising applying a voltage across an aqueous solution comprising the charged compound, the amphiphilic compound and a background electrolyte in an electroseparation syringe comprising an anode and a cathode positioned to apply the voltage across the aqueous solution.
   44. An electroseparation syringe, comprising:
a barrel having a discharge end and a receiving end;
a plunger;
a cathode comprising a first power supply connector; and
an anode comprising a second power supply connector,
   wherein the cathode and the anode are configured to provide a voltage across a solution contained in the barrel.
      45. The electroseparation syringe of item 44, wherein one of the cathode or the anode is positioned at the discharge end of the barrel.
      46. The electroseparation syringe of item 44 or 45, wherein the plunger comprises one of the cathode or the anode.
      47. The electroseparation syringe of any one of items 44 to 46, wherein the barrel comprises an internal coating.
      48. The electroseparation syringe of any one of items 44 to 47, comprising a valve located at the discharge end of the barrel.
      49. An analytical system, comprising:
an electroseparation syringe comprising an anode and a cathode positioned to apply a voltage across an aqueous solution contained in the electroseparation syringe;
a power supply configured to connect with the anode and the cathode; and
an analyser adapted to receive and analyse an analyte from the electroseparation syringe.
   50. A system, comprising:
a receiver for an aqueous solution injected from an electroseparation syringe comprising an anode and a cathode positioned to apply a voltage across the aqueous solution when contained in the electroseparation syringe, and
a power supply configured to connect with the anode and the cathode.
   51. A syringe comprising:
a syringe barrel,
a needle receiver at a discharge end of the syringe barrel adapted to receive a needle, and
a plunger comprising an electrode positioned within the syringe barrel, wherein the electrode is configured to come into electrical contact with a solution contained within the syringe barrel in use,
   wherein in use, a second electrode is positioned at a discharge end of the syringe barrel, and a voltage applied across the electrodes results in a voltage being applied longitudinally across the solution contained in the syringe barrel.
      52. The syringe of item 51, wherein the needle constitutes the second electrode.

1. A process for modifying the distribution of a compound in a solution comprising:
drawing the solution comprising the compound into an electroseparation syringe comprising a syringe barrel, a plunger and electrodes positioned to apply a voltage across solution contained in the syringe barrel, and
applying a voltage across the solution in the syringe barrel to modify the distribution of the compound within the solution contained in the syringe barrel.

2. The process of claim 1, wherein the compound is an analyte.

3. The process of claim 2, wherein the modifying of the distribution of the analyte within the solution contained in the syringe barrel comprises:
focusing a concentration of the analyte within a region of the solution contained in the syringe barrel, or
generating a region within the solution with an increased concentration of the analyte in the solution, wherein the analyte is a net neutral molecule; or
separating the analyte from net neutral compounds also contained within the solution, wherein the analyte is a charged compound.

4. The process of claim 1, wherein the solution comprises the compound and an analyte, and the modifying of the distribution of the compound within the solution contained in the syringe barrel comprises focusing an increased concentration of the compound within one region of the solution to create another region of solution containing the analyte with a depleted concentration of the compound.

5. The process of claim 2, comprising:
injecting the solution into an analyser to allow analysis of the analyte.

6. The process of claim 2, wherein the voltage applied to the solution is a voltage within the range of −1.23V to about −5000 V, or within the range of +1.23V to about 5000V.

7. The process of claim 2, wherein a second voltage is applied during the step of injecting the solution into the analyser.

8. The process of claim 7, wherein the second voltage is a voltage in a range of about −1.23V to about −1000V or within the range of about +1.23V to about +1000V.

9. The process of claim 2, wherein prior to applying the voltage across the aqueous solution, the pressure within a barrel of the electroseparation syringe is increased.

10. The process of claim 9, wherein the electroseparation syringe comprises a valve associated with a discharge end of the barrel, and the pressure is increased by applying pressure on the plunger of the electroseparation syringe when the valve is closed.

11. The process according to claim 2, wherein:
the analyte is an amphiphilic analyte, or
the analyte is a neutral analyte or a charged analyte, and the solution comprises an amphiphilic molecule.

12. The process according to claim 11, wherein the amphiphilic analyte or amphiphilic molecule is selected from a protein, a peptide and an amino acid.

13. The process of claim 1, wherein the solution comprises a background electrolyte selected from the group consisting of ammonium salts, carboxylic acids, carboxylate salts, amines and combinations of one or more thereof.

14.-16. (canceled)

17. An electroseparation syringe comprising a syringe barrel, a plunger, and a pair of electrodes, wherein the electrodes are configured to come into electrical contact with a solution contained within the syringe barrel in use, so as to enable a voltage to be applied longitudinally across solution contained in the syringe barrel.

18. The electroseparation syringe of claim 17, wherein the pair of electrodes comprise:
a cathode comprising a first power supply connector; and
an anode comprising a second power supply connector.

19. (canceled)

20. The electroseparation syringe of claim 17, wherein the plunger comprises one of the electrodes.

21. The electroseparation syringe of claim 17, wherein one of the electrodes is in the form of a needle.

22. (canceled)

23. The electroseparation syringe of claim 17, comprising a valve associated with the discharge end of the barrel.

24. The electroseparation syringe of claim 17, with a capacity of between 0.5 μl and 1 ml, such as between 0.5 μl and 20μ.

25.-27. (canceled)

28. An analytical system, comprising:
an electroseparation syringe comprising a syringe barrel, a plunger, and a pair of electrodes positioned to apply a voltage across an aqueous solution contained in the syringe barrel in use;
a power supply configured to connect with the electrodes; and
an analyser adapted to receive and analyse an analyte from the electroseparation syringe.

29.-30. (canceled)
